(19) United States
(12) Patent Application Publication
(10) Pub. No.: US 2024/0151728 A1
(43) Pub. Date: May 9, 2024

(54) FLUORESCENCE RESONANCE ENERGY TRANSFER-BASED BIOSENSOR FOR SENSING CHIMERIC ANTIGEN RECEPTOR ACTIVITY AND USE THEREOF

(71) Applicants: Korea Institute of Science and Technology, Seoul (KR); SEOUL NATIONAL UNIVERSITY R&db FOUNDATION, Seoul (KR)

(72) Inventors: Jihye SEONG, Seoul (KR); Mihue Jang, Seoul (KR); Haenim Lee, Seoul (KR); Hyejin Yoo, Seoul (KR); Chang-Han Lee, Namyangju-si (KR); Jiyun Jeong, Bucheon-si (KR); Hang-Rae Kim, Seoul (KR); Soojin Lee, Seongnam-si (KR)

(73) Assignees: Korea Institute of Science and Technology, Seoul (KR); SEOUL NATIONAL UNIVERSITY R&DB FOUNDATION, Seoul (KR)

(21) Appl. No.: 18/487,614

(22) Filed: Oct. 16, 2023

(30) Foreign Application Priority Data
Nov. 4, 2022 (KR) 10-2022-0146397
Jan. 20, 2023 (KR) 10-2023-0009038
Aug. 7, 2023 (KR) 10-2023-0103122

(51) Int. Cl. G01N 33/574; G01N 33/533; G01N 33/542; G01N 33/68

(52) U.S. Cl. CPC G01N 33/57492; G01N 33/533; G01N 33/542; G01N 33/6845; C12N 2503/00

(57) ABSTRACT

The disclosure relates to a fluorescence resonance energy transfer (FRET)-based biosensor for sensing chimeric antigen receptor (CAR) activity and use thereof, and in particular, to a FRET-based biosensor, which simultaneously detects binding of an antigen-binding receptor domain to a cancer antigen and consequent activation of T cells, and a method of screening for CAR activity in a live cell by using the FRET-based biosensor.

### CROSS-REFERENCES TO RELATED APPLICATION

This application is based on and claims priority under 35 U.S.C. § 119 to Korean Patent Application Nos. 10-2022-0146397, filed on Nov. 4, 2022, 10-2023-0009038, filed on Jan. 20, 2023, and 10-2023-0103122, filed on Aug. 7, 2023, in the Korean Intellectual Property Office, the disclosures of which are incorporated by reference herein in their entirety.

### INCORPORATION BY REFERENCE OF SEQUENCE LISTING

The content of the electronically submitted sequence listing, file name: Q291992_Sequence_Listing_ST26_AS_FILED.xml; size: 26,511 bytes; and date of creation: Oct. 13, 2023, filed herewith, is incorporated herein by reference in its entirety.

### BACKGROUND

### 1. Field

The disclosure relates to a fluorescence resonance energy transfer (FRET)-based biosensor for sensing chimeric antigen receptor (CAR) activity and use thereof, and in particular, to a FRET-based biosensor, which simultaneously detects binding of an antigen-binding receptor domain to a cancer antigen and consequent activation of T cells, and a method of screening for CAR activity in a live cell by using the FRET-based biosensor.

### 2. Description of the Related Art

Chimeric antigen receptor (CAR)-T cell therapy is a treatment involving preparing CAR-T cells by genetically engineering T cells from a patient to express CAR and infusing the CAR-T cells back into the patient's body. Such CAR-T cells selectively recognize cancer antigens and activate T cells to effectively kill cancer cells.

In the CAR regarded as the core of the mechanism of action of the CAR-T cell, a portion that recognizes a cancer antigen is present outside the cell membrane of the T cell, and a portion that can activate the T cell after recognizing the antigen is located inside the T cell. A CAR protein is designed in such a way that a single-chain variable fragment (scFv) of an antibody recognizing a cancer antigen is linked to intracellular signaling domain through a backbone (Dotti G, et al., Immunol Rev. 2014; 257(1):107-26). The intracellular signaling domain is mainly based on the intracellular signaling domain of the CD3 zeta (ζ) chain, which is a signaling subunit of the T cell receptor (first-generation CAR), and it has evolved to a form having added thereto the intracellular signaling domain of a co-stimulatory molecule that stimulates the growth and differentiation of the T cells. The first step in the action of a CAR-T cell prepared by introducing a vector including a CAR encoding nucleic acid into a T cell is for its scFv part to recognize a cancer antigen on the surface of a cancer cell. Therefore, screening of effective scFvs is critical to the success of CAR-T therapy.

The development of a CAR vector has focused on screening of scFvs mainly based on affinity for cancer antigens. In this regard, scFvs binding to cancer antigens are screened by using a phage display technique, and then screened by measuring binding affinities between the scFvs and the cancer antigens based on surface plasmon resonance (SPR). However, in such recombinant protein-based CAR screening conditions, the context of CARs such as expression structure on the cell surface and its surrounding environment cannot be considered. Thus, the screened CAR may not actually bind to cancer antigens expressed on the cell surface, or even if the CAR screened based on the binding affinity for cancer antigens binds to cancer antigens in the actual cellular environment, the binding may not lead to effective activation of the CAR-T cells. In addition, in the existing CAR screening, the actual environment around the cell surface or the spacer in CARs has not been sufficiently considered. For example, when a CAR is physically hidden by other larger receptors around cancer antigens, antigen binding may not occur if the length of a spacer in the CAR is short. On the other hand, if the length of the spacer is too long, an immune synaptic distance increases, resulting in a reduction in the effect. Thus, the length of the spacer in a CAR should be considered to ensure an effective contact of the CAR with the cancer antigen. Even after the scFv is screened, preparing and testing candidate CAR-T cells by expressing a CAR vector including the screened scFv in T cells are costly and time-consuming, and in many cases, the screened scFvs may be found having no or low CAR-T activity.

Therefore, to overcome limitations of the existing screening methods and successfully develop CAR vectors, there is a need for an integrated screening system that can simultaneously and precisely detect binding of a CAR to cancer antigens and consequent activation of CAR-T cell in a live cell.

In order to overcome limitations of the existing CAR screening, the inventors of the present disclosure have developed a fluorescence resonance energy transfer (FRET)-based system for sensing CAR activity that can comprehensively and precisely measure CAR's binding to cancer antigens and the subsequent activation of T cell in live cells, and verified the mechanism of action, thereby completing the present disclosure.

### SUMMARY

Provided is a FRET-based biosensor for sensing CAR activity.

Provided is a method of screening for CAR activity in a live cell by using the FRET-based biosensor for sensing CAR activity.

Provided is a high throughput screening (HTS) system by using the FRET-based biosensor for sensing CAR activity.

Additional aspects will be set forth in part in the description which follows and, in part, will be apparent from the description, or may be learned by practice of the presented embodiments of the disclosure.

An aspect of the disclosure provides a fluorescence resonance energy transfer (FRET)-based biosensor for sensing chimeric antigen receptor (CAR) activity, the biosensor comprising
an antigen-binding receptor domain, a transmembrane domain, an immunoreceptor tyrosine-based activation motif (ITAM) domain, a FRET domain comprising a fluorescence donor and a fluorescence acceptor, and a ZAP-SH2 domain,
wherein binding of a target antigen to the antigen-binding receptor domain leads to phosphorylation of the ITAM domain, and the phosphorylated ITAM domain binds to the ZAP-SH2 domain so that a detectable FRET signal is generated in the FRET domain, thereby simultaneously detecting antigen binding and consequent activation of T cells to verify CAR activity.

It is known that, after CAR-T cells recognize cancer cells through scFvs, signals generated therefrom are transmitted into the intracellular signaling portion to phosphorylate the ITAM of CD3, and an SH2 domain of a signaling substance called ZAP binds to the phosphorylated ITAM to initiate activation of T cells. This is also the mechanism of action for the T cell receptors associated with CD3. Accordingly, the inventors of the present disclosure developed a CAR vector verification prototype sensor designed to closely and accurately observe the phosphorylation of the ITAM upon binding of cancer antigen in a live cell environment, and the subsequent binding of the SH2 domain. Therefore, the biosensor according to one aspect of the disclosure is a biosensor for screening a CAR vector designed to very sensitively observe the phosphorylation of the ITAM upon binding of a cancer antigen in a live cell environment, and the subsequent binding of the SH2 domain. The FRET-based biosensor for sensing CAR activity may be referred to as the “CAR-FRET biosensor”, “biosensor” or “sensor” in the present disclosure.

As used herein, the term “antigen-binding receptor domain” refers to an extracellular binding domain that confers target specificity to a CAR molecule, and may be a scFv (single-chain fragment variable) which contains only variable heavy (VH) and variable light (VL) chains of an antibody joined together and can specifically bind to a target antigen. In the present application, this term is used interchangeably with “antigen-binding domain”.

As used herein, the term “transmembrane domain” refers to a domain that connects an antigen-binding receptor domain to an intracellular signaling domain to structurally stabilize sensor and transduce signals.

As used herein, the term “immunoreceptor tyrosine-based activation motif (ITAM) domain” is a domain including tyrosine that is phosphorylated when a ligand is bound to an immunoreceptor and activated. When the antigen-binding receptor domain binds to a target antigen, it is phosphorylated and then binds to a ZAP-SH2 domain, thereby generating FRET signals according to the antigen binding and activation of T cells. The ITAM domain may be derived from a zeta chain of CD3 protein.

As used herein, the term “FRET domain” refers to a domain including a fluorescence donor and a fluorescence acceptor arranged such that FRET occurs upon binding of a target antigen to the antigen-binding acceptor domain, subsequent phosphorylation of the ITAM domain, and binding of the phosphorylated ITAM domain to ZAP-SH2 domain. Here, signals representing the antigen binding and activation of T cells are generated based on the emission ratio of the fluorescence donor and the fluorescence acceptor.

As used herein, the term “ZAP-SH2 domain” refers a domain that binds to the phosphorylated ITAM domain after tyrosine residues in the ITAM domain are phosphorylated. When ZAP-SH2 domain binds to the phosphorylated ITAM domain, FRET signal is generated.

The biosensor according to the disclosure includes the antigen-binding receptor domain recognizing an antigen, which is connected to the transmembrane domain, the transmembrane domain transducing signals, which is connected to the ITAM domain of CD3, the ITAM domain being phosphorylated upon binding of a target antigen to the antigen-binding receptor domain, which is connected to a FRET domain, the FRET domain including a fluorescence donor and a fluorescence acceptor, which is connected to the ZAP-SH2 domain, and the ZAP-SH2 domain binding to the phosphorylated ITAM domain. The FRET domain includes a pair of a fluorescence donor protein and a fluorescence acceptor protein to generate a FRET signal depending on a state of the biosensor. In a state where an antigen is not bound to the biosensor, the fluorescence donor protein and the fluorescence acceptor protein exist at a distance and orientation at which FRET does not occur, whereas, in a state where an antigen is bound to the antigen-binding domain, leading to phosphorylation of the ITAM domain and then the ZAP-SH2 domain binds to the phosphorylated ITAM domain, the distance between a pair of the fluorescent proteins in the FRET domain becomes close enough to allow FRET to occur, thereby generating FRET signals. That is, FRET occurs only when both the antigen binding and consequent activation of T cells are achieved, whereas FRET does not occur when the antigen binding fails to lead to activation of T cells.

As used herein, the term “FRET” stands for fluorescence resonance energy transfer, and is generally referred to as resonance energy transfer because the wavelength emitted from the fluorescence donor overlaps with the absorption spectrum of the fluorescence acceptor and the energy transfer occurs without appearance of photons. The FRET is a result of the long-distance dipole-dipole interaction between the fluorescence donor and the fluorescence acceptor. The energy transfer efficiency of FRET may be determined by the overlapping range of the emission spectrum of the fluorescence donor and the absorption spectrum of the fluorescence acceptor, the quantum efficiency of the fluorescence donor, the relative orientation of the transition dipoles of the fluorescence donor and the fluorescence acceptor, and the distance between the fluorescence donor and the fluorescence acceptor.

As used herein, the term “fluorescence donor” is a fluorescent substance that acts as a donor in the FRET phenomenon, and refers to a donor of a fluorescence signal that transfers energy for FRET, and the term “fluorescence acceptor” is a fluorescent substance that acts as an acceptor in the FRET phenomenon.

Any substances may be used as long as the emission spectrum of the fluorescence donor and the absorption spectrum of the fluorescence acceptor overlap each other to cause FRET or fluorescence reduction. Therefore, as the fluorescence donor, fluorescent proteins and fluorescent dyes of various wavelengths, bioluminescent proteins, quantum dots, and the like may be used, and as the fluorescence acceptor, fluorescent proteins, fluorescent dyes, quantum dots, and the like, having different wavelengths from that of the fluorescent donor, may be used. Considering properties of the fluorescence donor and the fluorescence acceptor such as the extinction coefficient, quantum efficiency, photostability, and convenience of use, fluorescent proteins, e.g., enhanced cyan fluorescence protein (ECFP) and enhanced yellow fluorescent protein (EYFP), may be used in a FRET module.

The biosensor according to the disclosure may efficiently detect the CAR activity in a live cell by detecting FRET signals from the binding of CAR to a target antigen, subsequent signal transduction and the resulting activation of T cells.

A library of CARs, such as the FRET-based biosensor of the disclosure including various types of scFv sequences for target antigens is prepared and then expressed in cells, the CAR expressing cells are contacted with or co-cultured with cells expressing target antigens such as cancer antigens, so as to detect FRET signals, thereby efficiently screening CARs. The inventors of the present disclosure have found that various CAR-FRET biosensor variants according to the disclosure efficiently measure various activity levels through FRET signals induced upon binding to cancer antigen-expressing cells, and that the measured FRET signals showed correlation with signals of CD69, a T cell activation marker involved in signal transduction.

In an embodiment of the present disclosure, the biosensor may further include a spacer between the antigen-binding receptor domain and the transmembrane domain.

In an embodiment of the present disclosure, the spacer may be selected so that the antigen-binding receptor domain is accessible to a target epitope in the surface environment of cells expressing a target antigen.

The spacer may include a CD-derived hinge region and may vary in length and composition. To function as CAR-T cells, the CAR should recognize and bind to a target antigen, but despite having binding affinity, the CAR may fail to bind to the target antigen when the target antigen is physically hidden by other receptors in its vicinity. Since the interaction between cancer antigens and the CAR in the actual cell surface environment is affected by accessibility or the surrounding environment in addition to the binding affinity between cancer antigens and the CAR, the effect of CAR-T cells may be enhanced by adjusting the length of a spacer. The CAR-T cells may function effectively by increasing the length of a spacer to allow the binding to antigens which are inaccessible or difficult to access. Accordingly, when the biosensor according to the disclosure includes a spacer, the accuracy and sensitivity of screening for CARs for various cancer antigens may be improved.

As used herein, the term “hinge” is a structural region that connects the transmembrane domain and the extracellular antigen-binding receptor domain, and provides spatial flexibility so that the antigen-binding receptor domain may have a suitable length to access a target antigen or an epitope.

In an embodiment of the present disclosure, the spacer may be a hinge of CD8a, a hinge of CD28, or an Fc region of IgG.

In an embodiment of the present disclosure, the biosensor may further include a co-stimulatory domain between the transmembrane domain and the ITAM domain. The co-stimulatory domain may be CD28 or 4-IBB.

In an embodiment of the present disclosure, the ITAM domain may be derived from CD3 zeta chain.

In an embodiment of the present disclosure, the FRET domain may include a fluorescence donor protein and a fluorescence acceptor protein, wherein the fluorescence donor protein and the fluorescence acceptor protein may be connected to each other via a first linker, and the C-terminus of the FRET domain and the ZAP-SH2 domain may be connected to each other via a second linker.

In an embodiment of the present disclosure, the first linker and the second linker may be selected to increase the sensitivity of FRET signal detection of the biosensor.

In an embodiment of the present disclosure, the first linker and the second linker may be selected to maximize the FRET signals in the FRET domain according to the binding of the antigen-binding receptor domain to cancer antigens and consequent activation of T cells by signal transduction induced by the binding, i.e., the phosphorylation of the ITAM domain and the binding of the ZAP-SH2 domain to the phosphorylated ITAM domain.

In an embodiment of the present disclosure, the first linker and the second linker may be selected to have the highest change in FRET ratio according to the phosphorylation of the ITAM domain and subsequent binding of the ZAP-SH2 domain thereto.

In an embodiment of the present disclosure, the first linker may be an ER/K motif of SEQ ID NO: 18, the second linker may be a GSG(7) motif of SEQ ID NO: 6, the transmembrane domain may have an amino acid sequence of SEQ ID NO: 8, the ITAM domain may have an amino acid sequence of SEQ ID NO: 1, the FRET domain may include yellow fluorescent protein (YFP) of SEQ ID NO: 4 and cyan fluorescent protein (CFP) of SEQ ID NO: 5, and the ZAP-SH2 domain may have an amino acid sequence of SEQ ID NO: 2.

In an embodiment of the present disclosure, the antigen-binding receptor domain may be scFv for cancer antigens, and the FRET domain may include YFP and CFP, wherein, when the scFv binds to a cancer antigen, the ITAM domain is phosphorylated, and the ZAP-SH2 binds to the phosphorylated ITAM domain to generate FRET signals in the FRET domain, thereby detecting the binding of the cancer antigen and consequent activation of T cells.

In an embodiment of the present disclosure, the FRET signal may be measured by measured by a FRET ratio of the fluorescence donor and the fluorescence acceptor, or a change in the FRET ratio.

As used herein, the term “FRET ratio” refers to a ratio of the emission intensity of the fluorescence acceptor to the emission intensity of the fluorescence donor measured after stimulation with the excitation wavelength of the fluorescence donor. For example, when CFP (mTurquoise) is used as the fluorescence donor and YFP (mCitrine) is used as the fluorescence acceptor, the CFP may have a maximum excitation wavelength of 430 nm and a maximum emission wavelength of 476 nm, and the YFP may have a maximum excitation wavelength of 516 nm and a maximum emission wavelength of 529 nm. Here, the FRET ratio is calculated by the following formula:

FRET ratio=YFP_{529 nm}/CFP_{476 nm}
wherein YFP_{529 nm} represents emission of YFP measured at CFP excitation wavelength (430 nm); and
CFP_{476 nm} represents emission of CFP measured at CFP excitation wavelength (430 nm).

In an embodiment of the present disclosure, the biosensor may be present in a membrane of a live cell, and may be able to verify CAR activity in an actual cell environment by co-culture with a cell expressing a target antigen.

In an embodiment of the present disclosure, the biosensor may be present in the membrane of cells, such as in the membrane of HEK or Jurkat cells.

In an embodiment of the present disclosure, the biosensor may be present in the membrane of T cells.

In an embodiment of the present disclosure, the first linker may be ER/K (SEQ ID NO: 18).

In an embodiment, the second linker may be GSG(7) (SEQ ID NO: 6).

In an embodiment of the present disclosure, the spacer may be selected to generate FRET signals according to the binding of the antigen-binding receptor domain to a target epitope and subsequent phosphorylation of the ITAM domain and the binding of the ZAP-SH2 domain thereto.

In order for the CAR-T cells to recognize an antigen expressed on the surface of a cancer cell, a cell membrane portion of the T cell and a cell membrane portion of an antigen-presenting cancer cell should be in sufficiently strong contact, and this contact portion forms an immunological synapse. Depending on the environment on the cell surface of a target antigen, when the length of the spacer is short, the binding of the antigen-binding receptor domain to the antigen may not be achieved despite having the binding affinity, and when the length of the spacer is long, the binding of the antigen-binding receptor domain to the antigen may be achieved, but consequent activation of T cells may be weak. Therefore, for the efficacy of the CAR, the spacer is selected to enable effective binding to cancer antigens and induce consequent activation of T cells.

In an embodiment of the present disclosure, the biosensor may include the ITAM domain of SEQ ID NO: 1 and the ZAP-SH2 domain of SEQ ID NO: 2.

In an embodiment of the present disclosure, the antigen-binding receptor domain may be scFv for a cancer antigen, and the biosensor may provide a tonic signal of a CAR by detecting a level of scFv-induced activation of T cells in the absence of a cancer antigen. The level of the scFv-induced activation of T cells in the absence of a cancer antigen refers to tonic or chronic activation of T cells without binding of a ligand, and may be also referred as “tonic signal” herein.

For efficacy of the CAR-T cells, it is preferred that the level of activation of T cells according to the binding of a cancer antigen is high and the level of tonic activation in the absence of a cancer antigen is low.

In an embodiment of the present disclosure, the biosensor may be used to identify the expression level and distribution of the CAR vector, and predict a tonic signal of the CAR by measuring the FRET level in the absence of a cancer antigen.

In an embodiment of the present disclosure, the FRET-based biosensor may identify a CAR as being active, when a change in a FRET ratio is higher than a change in a FRET ratio in the absence of an antigen and is 10% or higher.

In an embodiment of the present disclosure, the FRET-based biosensor may identify a CAR as being active, when a change in a FRET ratio is higher than a change in a FRET ratio in the absence of an antigen and is 20% or higher or 30% or higher.

In an embodiment of the present disclosure, the biosensor may be in the form of a single fusion protein, or may consist of a FRET receptor module (R-part) including an antigen-binding receptor domain, an ITAM domain, and a fluorescence donor or fluorescence acceptor of a FRET domain, and a FRET-cytoplasmic module (C-part) including a fluorescence donor or fluorescence acceptor not included in the FRET receptor module and a ZAP-SH2 domain.

Another aspect of the present disclosure provides a nucleic acid encoding the aforementioned biosensor according to the present disclosure.

In an embodiment of the present disclosure, the biosensor may be encoded by one or more nucleic acids.

In an embodiment of the present disclosure, the nucleic acid may include a first nucleic acid molecule and a second nucleic acid molecule, wherein the first nucleic acid molecule encodes the FRET-receptor module of the biosensor including the antigen-binding domain, the spacer, the transmembrane domain, the ITAM domain, and one fluorescence protein of the FRET domain, and the second nucleic acid molecule encodes the FRET-cytoplasmic module of the biosensor including the remaining fluorescence protein of the FRET domain and the ZAP-SH2 domain. The biosensor may be an intermolecular CAR-FRET sensor, which is encoded by the nucleic acid encoding the FRET-receptor module and the nucleic acid encoding the FRET-cytoplasmic module, and formed by expression and combination of individual nucleic acids.

In an embodiment of the present disclosure, the biosensor may consist of the FRET-receptor module and the FRET-cytoplasmic module, and the nucleic acid encoding the biosensor may include the first nucleic acid molecule encoding the FRET-receptor module and the second nucleic acid molecule encoding the FRET-cytoplasmic module.

In an embodiment of the present disclosure, the first nucleic acid molecule and the second nucleic acid molecule may encode CAR-YFP, the FRET-receptor module and ZAP-SH2-CFP, the FRET-cytoplasmic module, respectively.

Another aspect of the present disclosure provides a vector comprising the nucleic acid encoding the aforementioned biosensor according to the present disclosure.

In an embodiment of the present disclosure, the vector may include a first vector and a second vector, wherein the first vector may include the first nucleic acid molecule encoding the FRET-receptor module and the second vector may include the second nucleic acid molecule encoding the FRET-cytoplasmic module.

In an embodiment of the present disclosure, the biosensor according to the disclosure may be encoded by two or more nucleic acid molecules, and the vector may be a plurality of vectors, each including at least one of the two or more nucleic acid molecules.

Another aspect of the present disclosure provides a cell expressing the aforementioned biosensor according to the disclosure.

In an embodiment of the present disclosure, the cell may include one or more vectors encoding the biosensor.

In an embodiment of the present disclosure, the cell may include the first vector and the second vector, wherein the first vector may include the first nucleic acid molecule encoding the FRET-receptor module and the second vector may include the second nucleic acid molecule encoding the FRET-cytoplasmic module.

In an embodiment of the present disclosure, the cell may be immune cells, such as T cells, natural killer (NK) cells, natural killer T (NKT) cells, or macrophages, but embodiments are not limited thereto.

In an embodiment of the present disclosure, the cell may be HEK293A cells for use in high-throughput screening (HTS).

In an embodiment of the present disclosure, the immune cells may be T cells.

In an embodiment of the present disclosure, the cell may be CAR-T cells, CAR-NK cells, CAR-NKT cells, or CAR-macrophages.

In an embodiment of the present disclosure, the cell may be selected from the group consisting of CD4+ T cells, CD8+ cytotoxic T lymphocytes (CTLs), gamma-delta T cells, tumor infiltrating lymphocytes (TILs), and T cells separated from peripheral blood mononuclear cells (PBMCs).

Another aspect of the present disclosure provides
a method of screening for a CAR vector in a live cell by using the aforementioned biosensor according to the disclosure, the method including:
expressing the biosensor in a cell;
contacting the biosensor with a target antigen; and
measuring a change in FRET signals.

In an embodiment of the present disclosure, the biosensor may include, for screening scFvs having binding affinity for a target antigen, a scFv library as the antigen-binding receptor domain.

As used herein, the term “scFv library” refers to a group of scFvs capable of specifically binding to various antigens and epitopes, and the biosensors including such an scFv library as the antigen-binding domain may constitute a biosensor library.

In an embodiment of the present disclosure, the expressing of the biosensor in the cell may be expressing the biosensor including an scFv library for a target antigen as the antigen-binding receptor domain, and may include: constituting an scFv library for a target antigen; preparing a vector including a nucleic acid encoding the biosensor including the scFv library as the antigen-binding receptor domain; and transfecting a cell with the vector.

In an embodiment of the present disclosure, the expressing of the biosensor may further include: constructing a spacer library including spacers of various lengths; and preparing a vector including a nucleic acid encoding the biosensor including the spacer library as the spacer. To select an optimal spacer for the biosensor that enables scFvs to effectively bind to a target antigen in a live cell environment, the biosensor including, as the antigen-binding receptor domain, scFvs screened for binding affinity for target antigens, a spacer library including spacers of various lengths and configurations is constructed, a biosensor library which includes the spacer library as the spacer is constructed and then the biosensor library is screened for the CAR activity.

In an embodiment of the present disclosure, the contacting of the biosensor with the target antigen may include: co-culturing the cell transfected with the vector including the nucleic acid encoding the biosensor and a cell expressing the target nucleic acid.

In an embodiment of the present disclosure, the measuring of a change in FRET signals may include selecting a CAR as an effective CAR when the change in FRET signals is higher than a change in a CAR verified to bind the target antigen and lead to activation of T cells.

In an embodiment of the present disclosure, when the change in FRET signals is higher than a FRET ratio measured in the absence of the target antigen, for example, 10% or more, the CAR may be selected as an effective CAR.

In an embodiment of the present disclosure, the measuring of a change in FRET signals may include selecting a CAR as a positive or effective CAR when the change in FRET signals is 10% or more and higher than a change FRET signals before the contacting of the biosensor with the target antigen.

In an embodiment of the present disclosure, the method may comprise preparing a vector encoding a biosensor including, as the antigen-binding receptor domain, an scFv library for a target antigen; transfecting the vector encoding the biosensor into a cell; contacting the transfected cell with an antigen; and measuring FRET signals, thereby screening scFvs for the target antigen, and may further include: preparing a vector encoding a biosensor library including, as the antigen-binding receptor domain, the screened scFvs, and as a spacer, a library encoding spacers of various lengths; transfecting into a cell with the vector encoding the biosensor library; contacting the transfected cell with the target antigen; and measuring FRET signals.

A biosensor library may be constructed by combining the scFvs screened by the FRET signals with various spacers, and then, a biosensor including a spacer that generates a desired FRET signal level by measuring the FRET signals upon binding to the antigen may be selected. Accordingly, an effective CAR vector that binds to cancer antigens in a live cell environment and induces activation of CAR-T cells to exhibit strong FRET signal values may be selected.

In an embodiment of the present disclosure, the method may be performed by live-cell imaging-based HTS. In particular, the method may be performed as HTS by detecting FRET signals after imaging cells expressing the biosensor according to the disclosure while co-culturing with cells expressing a target antigen.

In an embodiment of the present disclosure, the biosensor may be encoded by one vector to constitute an intramolecular CAR-FRET sensor.

In an embodiment of the present disclosure, the biosensor may be encoded by a receptor module (R-part) vector encoding a CAR-fluorescence protein and a cytoplasmic module (C-part) vector encoding a fluorescence protein-ZAP-SH2 domain, thereby constituting an intermolecular CAR-FRET sensor. Such an intermolecular CAR-FRET sensor may be used for HTS screening of the R-part library by using cells stably expressing the C-part.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 shows a schematic diagram of a fluorescence resonance energy transfer (FRET)-based chimeric antigen receptor (CAR) activity screening system according to an embodiment of the present disclosure. In order to screen for CARs by simultaneously detecting binding to a target cancer antigen and consequent activation of T cells, a sensor designed to generate FRET signals only when binding to a cancer antigen and consequent activation of CAR-T cells occur, is used. Specifically, when the antigen-binding receptor domain binds to a cancer antigen, the resulting signal is transmitted into the intracellular signaling domains, leading to phosphorylation of the ITAM domain and binding of the ZAP-SH2 domain to the phosphorylated ITAM domain, and accordingly, the distance between the fluorescence proteins in the FRET domain gets close to generate FRET signals.

FIG. 2 shows a schematic diagram of the structure and work of a biosensor according to an embodiment of the present disclosure. The biosensor includes: an antigen-binding receptor domain for binding to cancer antigens; an ITAM domain that is phosphorylated upon antigen binding; a FRET-signaling domain including a pair of a fluorescence donor and a fluorescence acceptor, which generates FRET by being brought closer by antigen binding, subsequent phosphorylation of the ITAM domain and binding of a ZAP-SH2 domain to the phosphorylated ITAM domain; and a ZAP-SH2 domain which binds to the ITAM domain that is phosphorylated upon the antigen binding. The biosensor may be expressed on the surface of live cells such as T cells, thereby generating FRET signals upon binding to antigens on the surface of cancer cells and consequent activation of T cells.

FIG. 3 shows the structure and work mechanism of a biosensor according to an embodiment of the present disclosure. The biosensor according to an embodiment consists of M5-specific scFv-CD8 hinge-CD8 transmembrane (TM) domain-4-1BB co-stimulatory domain and TM-CD3zeta-derived ITAM-YFP(mCitrine)-linker-CFP(mTurquiose2)-ZAP SH2, and is designed in such a way that, when scFv binds to an antigen M5, six tyrosine residues in the ITAM domain (2 tyrosine residues/ITAM domain) are phosphorylated, and the ZAP-SH2 binds to the phosphorylated tyrosines, thereby rendering the distance and orientation of YFP and CFP to generate FRET changes.

FIG. 4 shows FRET changes measured by co-culturing cells expressing a CAR-FRET biosensor according to an embodiment of the present disclosure (CAR cells) with either cell expressing mesothelin which is a target antigen of solid cancer (MSLN+) and cells not expressing mesothelin (MSLN−). Arrows indicate sites of cell-to-cell contact.

FIG. 5 shows fluorescence imaging and FRET measurement results obtained after treatment of wild-type (WT) and variant biosensors (ITAM^{YF}, ZAP-SH2^{RK}) according to an embodiment with pervanadate (PV).

FIG. 6 shows the results of western blotting obtained after treatment of the WT and variant biosensors (ITAM^{YF}, ZAP-SH2^{RK}) according to an embodiment with pervanadate.

FIG. 7 shows the structure of mesothelin which is a representative antigen of solid cancer, and the preparation of variants of CAR-FRET sensor including various scFvs recognizing epitopes of mesothelin, according to an embodiment of the disclosure.

FIG. 8 shows screening of scFvs of mesothelin by using a CAR-FRET sensor according to an embodiment of the present disclosure, wherein the FRET changes are detected by co-culturing cells expressing the CAR-FRET sensor including mesothelin epitope-recognizing scFv variants with cancer cells expressing mesothelin.

FIGS. 9A and 9B show the results of measuring CD69 signals to verify the correlation between the detection of FRET signals and the activation of T cells upon binding to cancer antigen in a CAR-FRET sensor according to an embodiment of the present disclosure.

FIG. 10 shows the results of measuring FRET signals (tonic signals) in the absence of cancer antigens by using a biosensor according to an embodiment of the present disclosure.

FIGS. 11A and 11B show the effect of substitution of a first linker and a second linker, respectively, in a biosensor according to an embodiment of the present disclosure, wherein the first linker connects fluorescence proteins in the FRET domain together, and the second linker connects a fluorescence protein in the FRET domain to a ZAP-SH2 domain.

FIGS. 12A and 12B show FRET changes over time and western blotting results detected by expressing a biosensor according to an embodiment of the present disclosure and biosensor variants in HEK 293 cells and co-culturing them with mesothelin-expressing cancer cells.

FIG. 13 shows FRET changes detected by expressing a biosensor according to an embodiment of the present disclosure and variants thereof, in H 293A cells and co-culturing them with mesothelin-expressing cancer cells.

FIG. 14 shows fluorescence imaging of comparison of biosensors according to an embodiment of the present disclosure, each comprising a different spacer, to identify an optimal spacer therein in detection of FRET signals by co-culture with cancer cells, according to the optimization of a spacer of a biosensor according to an embodiment.

FIG. 15 shows the results of detection FRET signals by co-culture with cancer cells, according to the optimization of a spacer of a biosensor according to an embodiment.

FIG. 16 shows FRET changes detected by expressing a biosensor according to an embodiment of the present disclosure (WT) and variants thereof (ITAM^{YF}, ZAP-SH2^{RK}) in Jurkat cells and co-culturing them with cancer cells. (A) Representative cell images; and (B) the graph of the FRET ratio changes of each group (%).

FIG. 17 shows western blot results showing the binding of a CAR-FRET sensor according to an embodiment of the present disclosure (WT) and variants thereof (ITAM^{YF}, ZAP-SH2^{RK}) to cancer antigens.

FIG. 18A shows a schematic diagram of a screening method based on a live FRET cell imaging-based CAR activity HTS (high-throughput screening) using a CAR-FRET sensor according to an embodiment of the present disclosure, and FIG. 18B shows quantitative relationship between expression and fluorescence of cancer antigens, which serves as the basis of the HTS system and quantification of FRET ratios in co-culture of a CAR-FRET sensor expressing cells with cancer antigen-expressing cells.

FIG. 19 shows an intermolecular CAR-FRET design in which each of fluorescence proteins in a FRET domain are present in a different molecule and thus, two molecules, each having a member of a pair of fluorescence proteins are combined to form a complete FRET sensor.

FIG. 20 shows changes in FRET ratio according to the length of a linker connecting a FRET-receptor module (R-part) and a FRET-cytoplasmic module (C-part) in an intermolecular CAR-FRET sensor according to an embodiment of the present disclosure.

FIG. 21 shows fluorescence imaging of co-culture of live cells expressing intermolecular CAR-FRET sensor according to an embodiment of the present disclosure and MSLN (mesothelin) expressing cells.

FIG. 22 shows a schematic diagram of a live cell imaging-based HTS system using a CAR-FRET sensor according to an embodiment of the present disclosure, and results of co-culture with cancer antigens.

FIG. 23 is a graph showing the expression level and binding affinity of M5 variants having diverse affinities for mesothelin. Here, NC represents a negative control group, and PC represents a positive control group, M5 scFv (DFDY).

FIG. 24 shows the results of measuring the affinity of wild-type M5 scFv (DFDY) and seven M5 scFv variants (DVAY, GVAD, DGDY, DGDD, GVDD, DGAD, and GCAY) for mesothelin.

FIG. 25 shows (A) fluorescence imaging and (B) changes in FRET ratio in co-culture of HEK293 cells expressing M5 scFv variants according to an embodiment of the present disclosure and HEK293A cells expressing human MSNL (hMSLN).

FIG. 26 shows (A) fluorescence imaging and (B) changes in FRET ratio in co-culture of Jurkat cells expressing M5 scFv variants according to an embodiment of the present disclosure and cells expressing hMSLN.

FIG. 27 shows the correlation between the detection of FRET signals by a CAR-FRET biosensor including M5 scFv variants according to an embodiment of the present disclosure and signals of CD69, a T cell activation marker.

FIGS. 28A, 28B, and 28C shows the results of measuring the affinity for hMSLN of M5 scFv DFDY according to an embodiment of the present disclosure and variants thereof, i.e., DVAY and GVDD, respectively.

FIGS. 29A and 29B show the results of measuring the cytokine productivity and apoptotic ability of M5 scFv DFDY according to an embodiment of the present disclosure and variant thereof, i.e., DVAY.

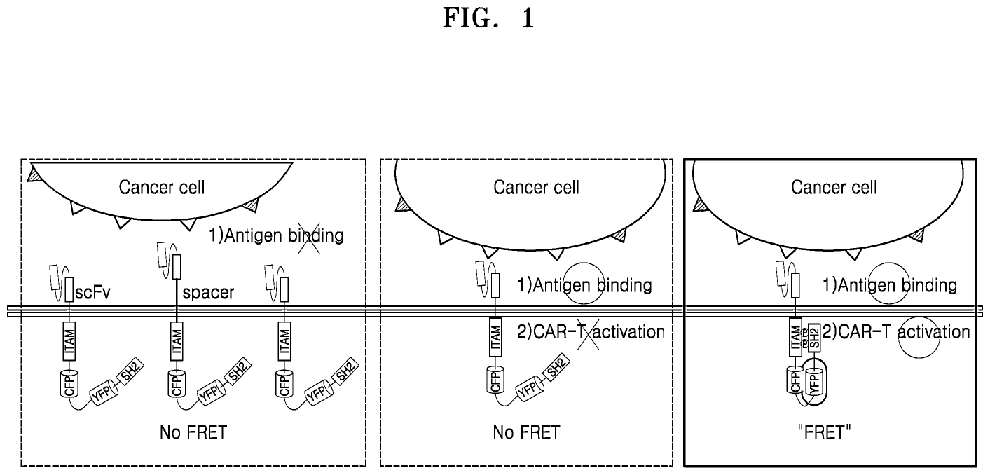

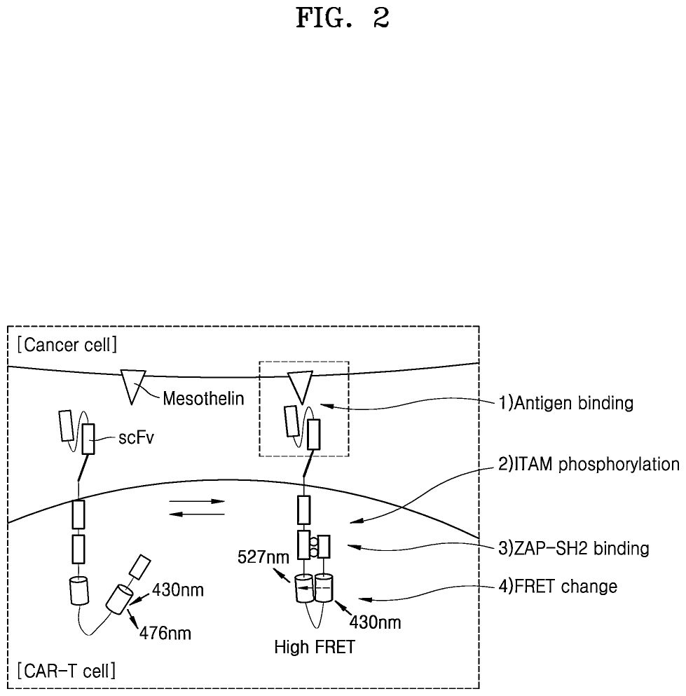

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

One or more embodiments of the present disclosure will be described in detail with reference to the following examples. However, these examples are for illustrative purposes only and are not intended to limit the scope of one or more embodiments of the present disclosure.

### Example 1. Design and Validation of FRET-Based CAR Activity Biosensor

(1) Design of CAR-FRET Biosensor

For efficient screening of chimeric antigen receptor (CAR) activity, a fluorescence protein-based FRET biosensor capable of simultaneously monitoring antigen affinity of CAR and consequent T cell activation was designed and produced.

The CAR-FRET biosensor is structured to detect cancer antigen binding and consequent T cell activation by comprising a CAR receptor domain capable of binding to a cancer antigen, an ITAM domain capable of being phosphorylated upon CAR binding to a cancer antigen, a FRET domain comprising a pair of fluoscence proteins, and a ZAP-SH2 domain capable of binding to a phosphorylated ITAM domain so that the antigen binding leads to phosphorylation of the ITAM domain, binding of the ZAP-SH2 domain to the phosphorylated ITAM domain, thereby the distance between fluoscence proteins in the FRET domain gets close to generate FRET signal.

In this Example, a FRET-based MSLN-CAR activity sensor was designed as the following structure in which MSLN(M5)-CAR consisting of scFv (M5) binding to mesothelin which is an important antigen expressed in solid tumors, a spacer consisting of a CD8 hinge portion, a CD8 transmembrane(TM) domain, a 4-1BB co-stimulatory domain, and a CD3 ITAM domain capable of initiating the function of TCR, a FRET domain of YFP(mCitrine)-linker-CFP(mTurqouoise2), a linker, and a ZAP-SH2 domain are combined was designed:

Leader sequence(L)-M5 scFv-hinge-TM-4-1BB-CD3ZETA-linker-YFP(mCitrine)-linker-CFP(mTurqouoise2)-linker-ZAP-SH2

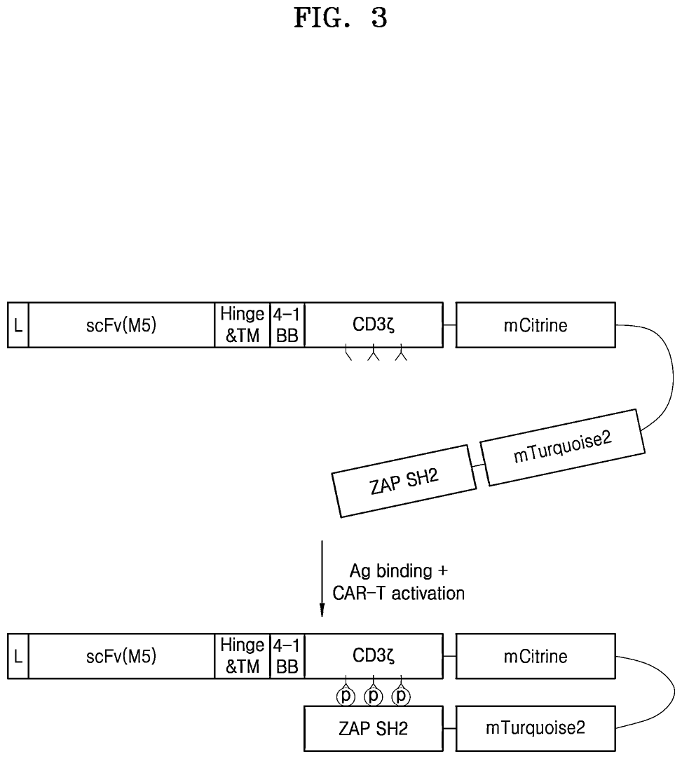

FIG. 3 shows the structure of the CAR-FRET sensor.

Specifically, the CAR-FRET DNA, a nucleic acid encoding the CAR-FRET sensor (SEQ ID NO: 11), was synthesized in vitro and inserted into a mammalian cell expression vector to prepare a CAR-FRET vector, wherein the CAR-FRET DNA consists of polynucleotides each encoding: a leader sequence (SEQ ID NO: 12); M5 scFv (SEQ ID NO: 10); CD8 hinge (SEQ ID NO: 7); CD8 transmembrane domain (SEQ ID NO: 8); 4-1BB (SEQ ID NO: 9); CD3zeta (ITAM) (SEQ ID NO: 1); linker (SEQ ID NO: 16); YFP (SEQ ID NO: 4); linker 1 (SEQ ID NO: 3); CFP (SEQ ID NO: 5); linker 2 (SEQ ID NO: 6); and ZAP-SH2 (SEQ ID NO: 2). The CAR-FRET vector was expressed in HEK293A cells by transfection with lipofectamine or in Jurkat cells by electroporation.

(2) Verification of CAR-FRET Biosensor Design

The CAR-FRET biosensor in this Example was designed in such a way that FRET would not be observed when it does not bind to a cancer antigen or its binding to a cancer antigen fails to induce activation of CAR-T cells. In this regard, the biosensor can simultaneously detect the binding of a cancer antigen and consequent activation of T cells. Only when the binding of a cancer antigen and consequent T cell activation are achieved, the biosensor will generate a positive FRET signal. The action of the biosensor was confirmed by co-culture of cells expressing the biosensor with cells expressing a cancer antigen.

(2-1) Co-Culture with Mesothelin-Expressing Cells

HEK293 and HEK293A cells were seeded in a cell culture dish at a density of 1×10⁵ cells/ml, respectively, and then cultured at 37° C. and 5% CO₂ for about 24 hours. HEK293 cells were used to express the CAR-FRET sensor, while HEK293A cells were used to express Mesothelin (MSLN), a cancer antigen. For transfection, 1 μg of the CAR-FRET sensor expression vector (also referred to as CAR vector) prepared in Section (1) and lipofectamine 2000 mixture was added to the HEK293 cells, while 1 μg of hMSLN-mCherry expression vector and lipofectamine 2000 mixture was added to the HEK293A cells. The cells were cultured for about 6 hours at 37° C. and 5% CO₂ for transfection. Afterwards, the HEK293 cells expressing the CAR-FRET sensor were washed with PBS, and the HEK293A cells expressing hMSLN-mCherry were attached to the dish coated with fibronectin and cultured overnight.

On the next day, for co-culture imaging, the HEK293 cells expressing the CAR-FRET sensor were inoculated into the culture dish of the HEK293A cells expressing hMSLN-mCherry, and then, images were obtained by real-time FRET imaging at 37° C. and 5% CO₂.

After removing the background signal by using the NIS program (Nikon), the fluorescence intensities of the ECFP and FRET channels were measured at the synapse where the CAR-FRET sensor-expressing cells and the hMSLN-expressing cells were bound to each other, and the FRET/ECFP ratio was obtained therefrom. The FRET/ECFP ratio for each group was calculated and quantified, and the difference in the FRET/ECFP ratio was graphed.

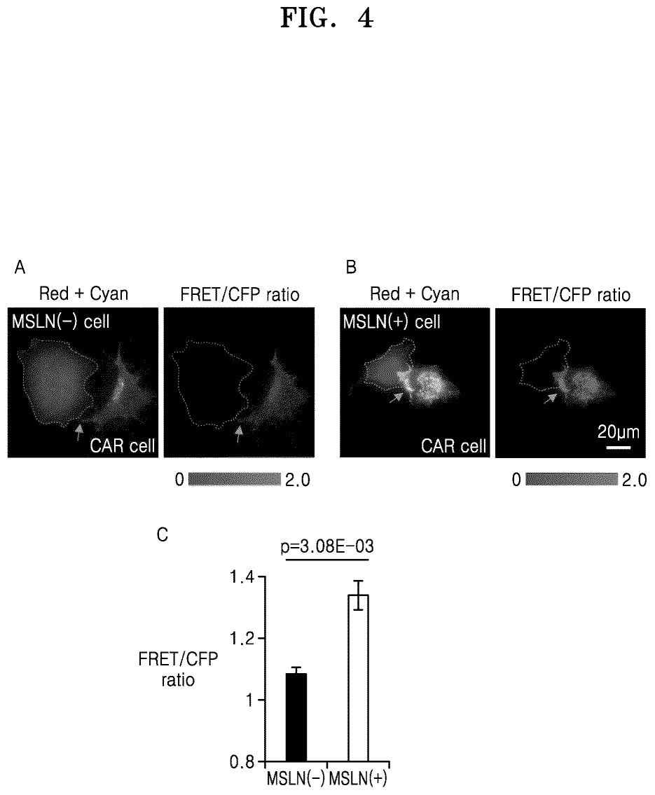

The results are shown in FIG. 4. In FIG. 4, (A) and (B) show the fluorescence imaging of the co-culture with HEK293A cells not expressing MSLN (MSLN(−)_cells) and HEK293A cells expressing MSLN (MSLN(+) cells), respectively, and C shows comparison of the FRET/CFP ratio therebetween. It was found that the FRET change (FRET/CFP) in co-culture with the mesothelin-expressing cells (MSLN+) was significantly higher than that in co-culture with mesothelin-expressing cells (MSLN−). In the co-culture of the mesothelin-expressing cells and the CAR vector-expressing cells, binding between mesothelin and scFv of CAR was achieved, and was followed by phosphorylation of the ITAM domain and binding of the ZAP-SH2 domain thereto and FRET generation between the fluorescence proteins, thereby leading to detection an increase in FRET signals (FRET/CFP ratio).

(2-2) Verification of Work Mechanism by Using Variants of CAR-FRET Sensor

In order for the CAR-FRET sensor of Section (1), which was designed to generate FRET signals by detecting binding to cancer antigens and consequent activation of T cells, to work and lead to a change in the FRET ratio, the antigen-binding receptor domain of the CAR-FRET sensor binds to a target antigen, and the subsequent signal transduction results in 1) phosphorylation of six tyrosine residues in the CD3ζ-ITAM domain, and 2) binding of R37 and R190 residues of the ZAP-SH2 domain to the phosphorylated CD3ζ-ITAM domain to make the distance between the fluorescence donor protein (e.g., CFP) and the fluorescence acceptor protein (e.g., YFP) close enough to cause FRET. Table 1 below shows the sequence of the CD3ζ-ITAM domain with residues to be phosphorylated, and the sequence of the ZAP-SH2 domain with residues to bind to the phosphorylated CD3ζ-ITAM domain.

TABLE 1

| | Sequence (SEQ ID NO) | Description |
|---|---|---|
| CD3ζ- | RVKFSRSADAPAYKQGQNQLYNELNLGRR | The underlined parts |
| ITAM | EEYDVLDKRRGRDPEMGGKPRRKNPQEGL | represent ITAM1, |
| | YNELQKDKMAEAYSEIGMKGERRRGKGHD | ITAM2, and ITAM3, |
| | GLYQGLSTATKDTYDALHMQALPPR (SEQ | respectively, and |
| | ID NO: 1) | bolded letters in each |
| | | part represent |
| | | residues to be |
| | | phosphorylated |
| ZAP-SH2 | PDPAAHLPFFYGSISRAEAEEHLKLAGMAD | This sequence |
| | GLFLLRQCLRSLGGYVLSLVHDVRFHHFPIE | corresponds to |
| | RQLNGTYAIAGGKAHCGPAELCEFYSRDPD | positions 2 to 258 of |
| | GLPCNLRKPCNRPSGLEPQPGVFDCLRDA | the ZAP70 amino acid |
| | MVRDYVRQTWKLEGEALEQAIISQAPQVEK | sequence (UniProt |
| | LIATTAHERMPWYHSSLTREEAERKLYSGA | Accession No. |
| | QTDGKFLLRPRKEQGTYALSLIYGKTVYHYL | P43404), and bold |
| | ISQDKAGKYCIPEGTKFDTLWQLVEYLK | letters represent R37 |
| | LKADGLIYCLKEACPNSS (SEQ ID NO: 2) | and R190 residues |
| | | which bind to the |
| | | phosphorylated CD3 |
| | | ITAM domain) |

To verify the work mechanism, 1) an ITAM^{YF} variant in which the tyrosine (Y) residue in the ITAM domain was substituted with phenylalanine (F) and 2) a ZAP-SH2^{RK} variant in which R37 and R190 residues in the ZAP-SH2 domain were substituted with lysine (K) were prepared. By using the wild-type (WT) CAR-FRET vector designed in Section (1) and the CAR-FRET vectors (ITAM^{YF} and ZAP-SH2^{RK}) including the variant ITAM domain and the variant ZAP-SH2 domain, respectively, the work mechanism of the CAR-FRET vector was verified.

Pervanadate (PV) Live Cell Imaging

HEK293 cells were seeded in a cell culture dish at a density of 1×10⁵ cells/ml, and then cultured at 37° C. and 5% CO₂ for about 24 hours. Then, for transfection, 1 μg of the CAR-FRET sensor expression vectors each containing the WT, the ITAM^{YF}, and the ZAP-SH2^{RK} were added to antibiotics-free Opti-MEM medium (ThermoFisher Scientific) and the resulting medium and lipofectamine 2000 (Invitrogen) mixture were added to the washed HEK293 cells. Then, the HEK293 cells were transfected by culturing at a temperature of 37° C. and 5% CO₂ for about 6 hours. At the end of transfection, the obtained cells were washed with PBS and cultured overnight.

On the next day, the HEK293 cells transfected with the CAR-FRET sensor expression vector were subcultured on fibronectin (10 μg/ml) coated glass-bottom dishes at a density of 1×10⁵ cells/ml and incubated for 2 hours for adhesion. FRET changes were detected by artificially inducing phosphorylation of the ITAM domain by treatment with pervanadate (PV) which is a phosphatase inhibitor.

To observe intracellular FRET changes upon the PV treatment, real-time cell imaging was performed at 37° C. and 5% CO₂ using a Nikon Ti-E inverted microscope. PV was prepared as previously described (Huyer G etal., JBC 1997, doi.org/10.1074/jbc.272.2.843), and was used within 24 hours after preparation. To detect the intracellular FRET changes, 438DF24 excitation filter, 458DRLP dichroic mirror, and 438DF24 (ECFP) and 542DF27 (FRET) emission filters were used to obtain images for 10 minutes at 1 minute intervals and after treatment with 100 μM PV, for 30 minutes at 1 minute intervals.

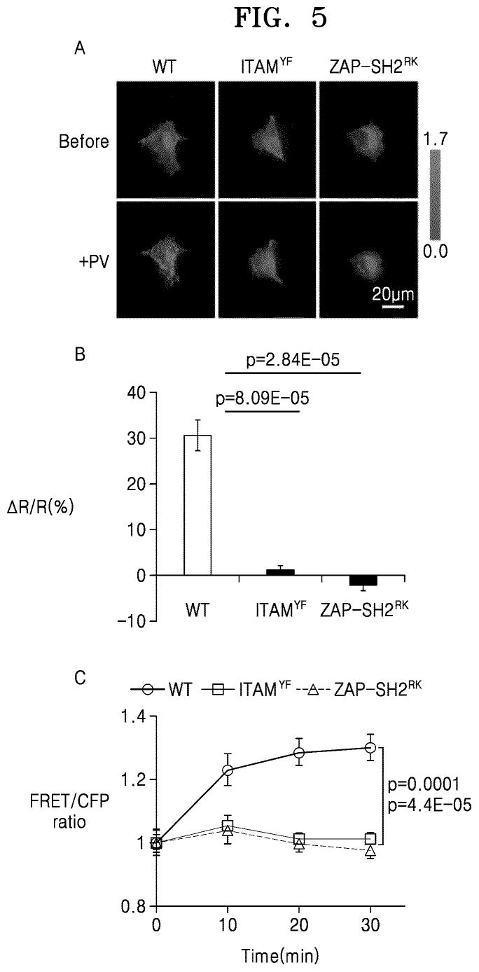

After removing the background signal by using the NIS program (Nikon), the fluorescence intensities of the ECFP and FRET channels were measured from the transfected cells, and the FRET/ECFP ratio was obtained therefrom. The FRET/ECFP ratio over time for each group was calculated and quantified, and the changes in AR/R (%) and FRET/ECFP ratio were graphed. The results are shown in FIG. 5. A shows a fluorescence imaging, and B and C show AR/R (%) and FRET/ECFP ratio, respectively. In cells transfected with the wild-type (WT) CAR-FRET biosensor, phosphorylation was induced by the PV treatment so that the FRET ratio was significantly increased, whereas, in cells transfected with the variants, ITAM^{YF} and ZAP-SH2^{RK}, in which a mutation was introduced to a site related to phosphorylation of the ITAM domain and consequent binding, there was no change in the FRET ratio.

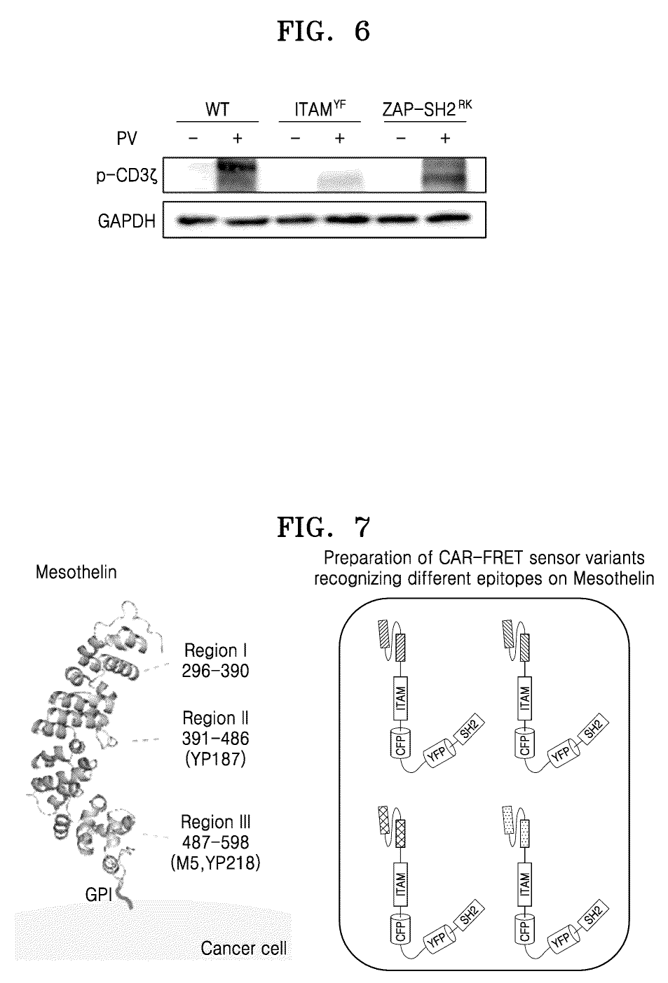

In addition, it was confirmed by western blotting that the PV treatment phosphorylated the CD3 zeta-derived ITAM domain in the wild-type CAR-FRET sensor and the ZAP-SH2^{RK} variant, but did not phosphorylate the ITAM^{YF} variant in which tyrosine for phosphorylation in the ITAM domain is substituted. Specifically, the HEK293 cells expressing the sensor and the HEK293A cells expressing hMSLN-mCherry were co-cultured. After extracting proteins by time, the amount of the proteins was quantified by BCA analysis. For western blotting, SDS-PAGE was performed by using 20 μg of each protein, and blots were added to a primary antibody solution containing anti-phospho CD3 (cell signaling, #67747) and anti-GAPDH (Santacruz, #5c47724) and incubated in a stirrer overnight at 4° C. On the next day, the resulting blots were washed using TBS-T buffer three times for 15 minutes each, and were added to a secondary antibody solution and incubated in a stirrer for 2 hours at room temperature. Then, the blots were washed using TBS-T buffer three times for 15 minutes each, and signals therefrom were detected by using an ECL solution. The results are shown in FIG. 6.

### Example 2. Optimization of FRET-Based CAR Biosensor

By using the FRET-based CAR biosensor (CAR-FRET biosensor) designed and validated in Example 1, an scFv targeting mesothelin, a linker, and a spacer were optimized.

By preparing CAR-FRET biosensor variants recognizing various mesothelin epitopes, scFvs leading to high activation of T cells were screened based on FRET signal changes were selected. Then, as linkers may affect the distance and orientation between the fluorescence proteins in the FRET domain thereby affecting FRET signals and a spacer may affect binding to antigens in the actual in vivo environment, in order to further increase the sensitivity of the FRET-based biosensor, variants with modified linkers and spacers were prepared to screen the linker and spacer for high sensitivity of the biosensor, i.e., high FRET signals.

(1) scFv

Mesothelin (MSLN) expressed on the surface of cancer cells in solid cancer is composed of three regions: region I (296-300), region II (391-486), and region III (487-598), and there are scFvs recognizing each region, such as M5, YP218, and YP187 (SEQ ID NO: 16). To screen scFvs that effectively recognize mesothelin and consequently leads to high CAR activity, CAR sensor variants were prepared by replacing the scFv portion of the CAR-FRET biosensor vector having the M5 scFv as prepared in Example 1 with a scFv recognizing different epitopes on mesothelin, and FRET signals were measured by co-culturing cells expressing the CAR-FRET sensor variant and cells expressing mesothelin.

FIG. 7 shows a schematic diagram of mesothelin and the preparation of CAR-FRET sensor variants recognizing different epitopes on mesothelin.

scFv Variants

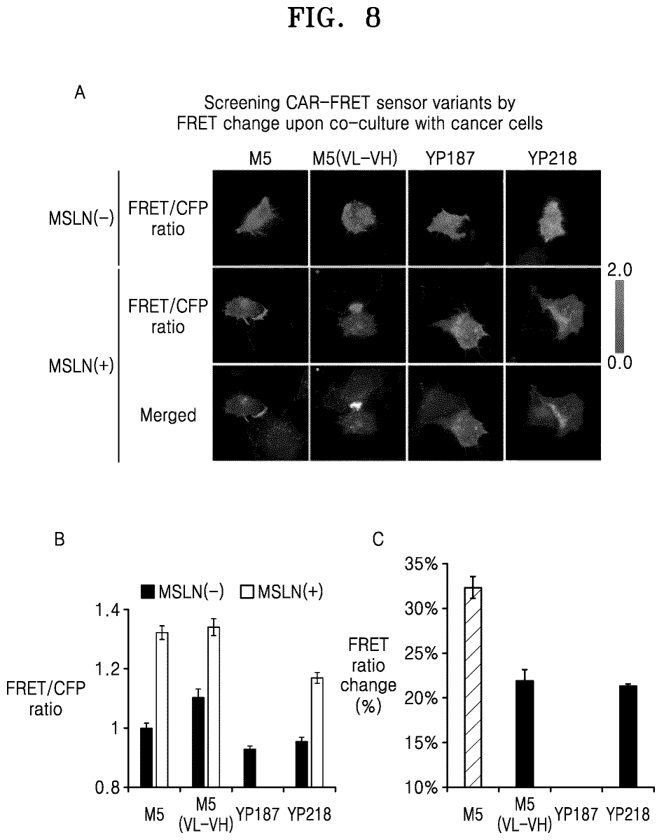

CAR-FRET vectors each including as an antigen-binding receptor domain M5 scFv (SEQ ID NO: 10), M5 (VL-VH) scFv (SEQ ID NO: 14), YP187 scFv (SEQ ID NO: 16), or YP218 scFv (SEQ ID NO: 15) were prepared and used to screen the CAR efficiency in live cells by measuring a change in FRET ratio in co-culture with cells expressing the cancer antigen. As described in Example 1, the FRET change was detected by performing real-time imaging in the co-culture with the cells expressing mesothelin. The results are shown in FIG. 8. In FIG. 8, (A) shows the fluorescence imaging and B and C show FRET/CFP ratio and FRET ratio change (%), respectively. Among epitopes targeting a membrane-proximal region of mesothelin, region III (487-598), the highest change in the FRET ratio was found in the CAR including M5 scFv.

Correlation with CD69 Signal

In addition, to verify whether the detection of FRET signals by the CAR-FRET biosensor reflects the activation of T cells according to the binding to cancer antigens, a CAR variant was prepared by removing the fluorescence protein and the linker for measuring FRET signals. The CAR variants were used to check the correlation with CD69 signal, a marker of the T cell activity, which is important for signal transduction. CAR-Jurkat cells including various scFvs of mesothelin were co-cultured with cells overexpressing cancer antigens, and then, CD69 signals measured therefrom were compared with FRET signals.

Lentivirus to be introduced into each Jurkat cell was prepared as follows. HEK293T cells were transfected with a shuttle vector, a psPAX2 vector, and a pMD2G vector at a ratio of 1:0.3:0.2, and then cultured for 2 days. On Day 1 and Day 2 after the transfection, the virus culture was obtained. Then, the virus culture was centrifuged at 1,350 rpm for 3 minutes and purified with a 0.45 μm filter before use. To prepare CAR-Jurkat cells by transduction, 10 ml of the virus culture medium together with polybrene (8 μg/ml) were added to 4×10⁴ Jurkat cells. RPMI medium was replaced 24 hours after the transduction. Jurkat cells into which a vector encoding the CAR variant from which the fluorescence protein and the linker were removed were co-cultured with cancer cells (K562 or MSLN-K562) expressing mesothelin at a ratio of 1:1 or 2:1. After 4 hours of the co-culture, all the cells were obtained and centrifuged at 1,350 rpm for 3 minutes. Afterwards, the cells were washed twice with PBS under the same conditions, and a staining process for FACS analysis was performed. To confirm the CD69 reactivity in the Jurkat cells, the cells were stained with a solution containing viability dye eFluor 506 (1:2500) and PE-Cy7 CD69 (5 μl/1M) at 4° C. for 20 minutes, and then washed twice with PBS as described above. The stained cells were first subjected to FCS/SSC gating to determine a singlet population, followed by FACS analysis at 780/60 wavelengths using a 488 nm blue laser.

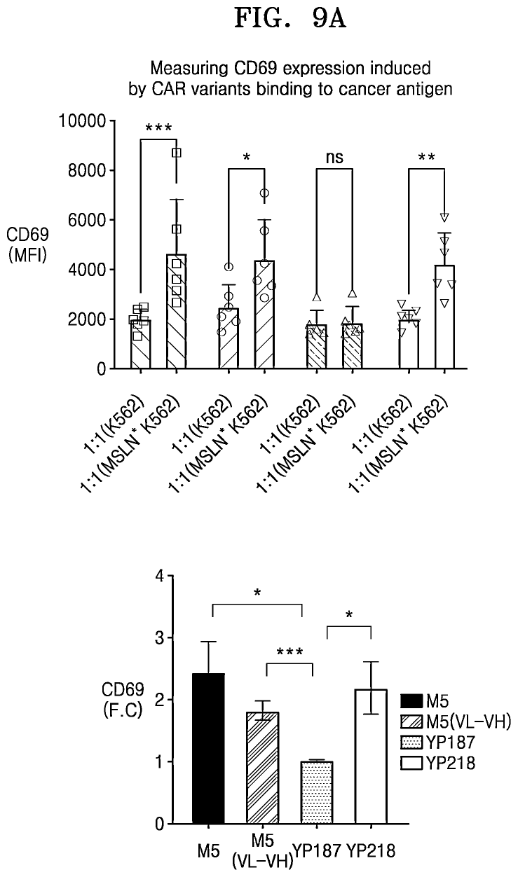

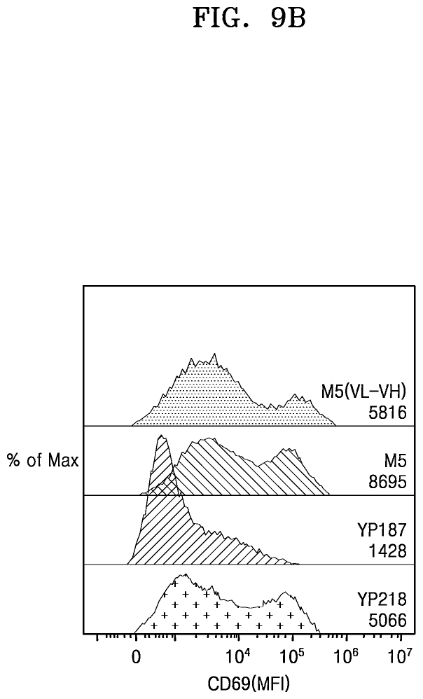

The results are shown in FIGS. 9A and 9B. The detection of FRET signals by the CAR-FRET biosensor was found to be quite similar to the expression of CD69, a marker of the T cell activation. Therefore, it was verified that live FRET-based CAR biosensors can be used to screen various CAR variants.

Detection of Tonic Signals

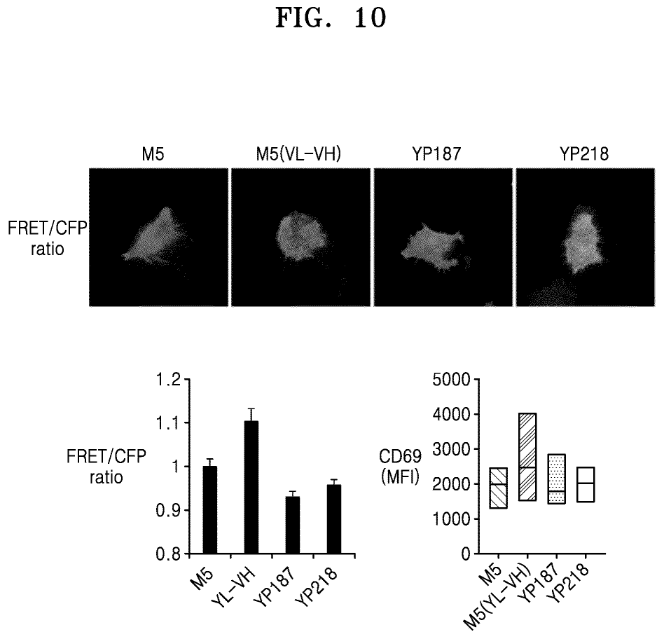

To develop an effective CAR-T cell therapeutic agent, it is necessary to select a CAR that has the maximum reactivity with cancer antigens, and at the same time, the minimum tonic signal. The tonic signal represents the baseline level of activation, which is always present in cells even in the absence of cancer antigens. The CAR-FRET sensor according to the present disclosure can measure tonic signals reflecting the activation of T cells in the absence of cancer antigens. The CAR-FRET biosensors including scFvs each recognizing various epitopes of mesothelin were expressed in Jurkat cells, and then levels of T cell activation in the absence of cancer antigens were detected as the FRET ratio (FRET/CFP ratio) and CD69 signals. The results are shown in FIG. 10.

In the absence of cancer antigens, it was confirmed that the values obtained by analyzing the FRET signals of cells expressing the FRET-based CAR sensor variant were coincident with the CD69 signals of the Jurkat cells. Therefore, the CAR-FRET biosensor can predict the degree of tonic activity of the CAR variants based on the FRET values.

(2) Linker

As shown in FIG. 3, the FRET-based CAR sensor includes a linker connecting the CD3zeta ITAM domain and the FRET domain, specifically, the first fluorescence protein in the FRET domain, a linker connecting the fluorescence donor protein and the fluorescence acceptor protein in the FRET domain, and a linker connecting the second fluorescence protein in the FRET domain and the ZAP-SH2 domain. Among these linkers, a first linker between fluorescence proteins directly affecting the generation of FRET signals and a second linker connecting a fluorescence protein in the FRET domain with the ZAP-SH2 domain were modified, and a consequent effect on the FRET signals was analyzed.

First Linker (Linker 1)

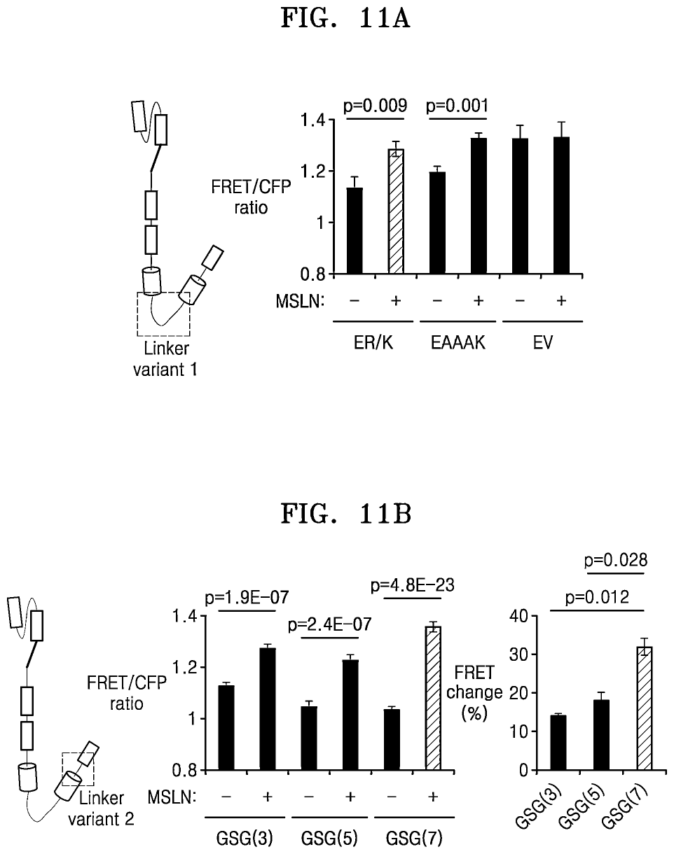

FRET-CAR vector linker variants including, as the first linker between the fluorescence proteins, an EAAAK linker, an ER/K linker, or an EV linker were prepared, and cells transfected with the FRET-CAR vector linker variants were co-cultured with cells expressing cancer antigens to measure a change in the FRET ratio. As a result, the highest change in the FRET ratio was detected when the ER/K linker was used as the first linker. The co-culturing and measuring of a change in the FRET ratio were performed as described in Example 1. The results are shown in FIG. 11A.

The structures of EAAAK linker, ER/K linker, and EV linker are as follows.

TABLE 2

| Name | Sequence (SEQ ID NO) |
|---|---|
| EAAAK | (GSG)₆(EAAAK)₆A(EAAAK)₆A(GSG)₆ |
| linker | (SEQ ID NO: 17) |
| ER/K | EEEEKKKQQEEEAERLRRIQEEMEKERK |
| linker | RREEDEERRRKEEEERRMKLEMEAKRKQ |
| | EEEERKKREDDEKRKKK |
| | (SEQ ID NO: 18) |
| EV linker | (SAAG)n |

Second Linker (Linker 2)

In addition, FRET-CAR vector linker variants including, as the second linker connecting a fluorescence protein in the FRET domain with the ZAP-SH2 domain, GSG(3), GSG(5), or GSG(7) were prepared, and cells transfected with the FRET-CAR vector linker variants were co-cultured with cells expressing cancer antigens to measure a change in the FRET ratio. As a result, the highest change in the FRET ratio was found when the second linker was GSG (7) (SEQ ID NO: 6). The results are shown in FIG. 11B.

CAR-FRET Biosensor Including Selected Linkers

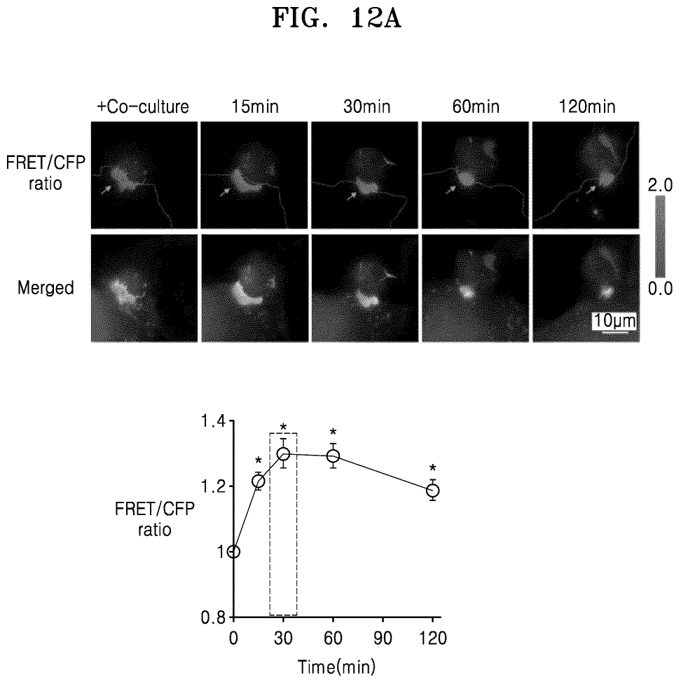

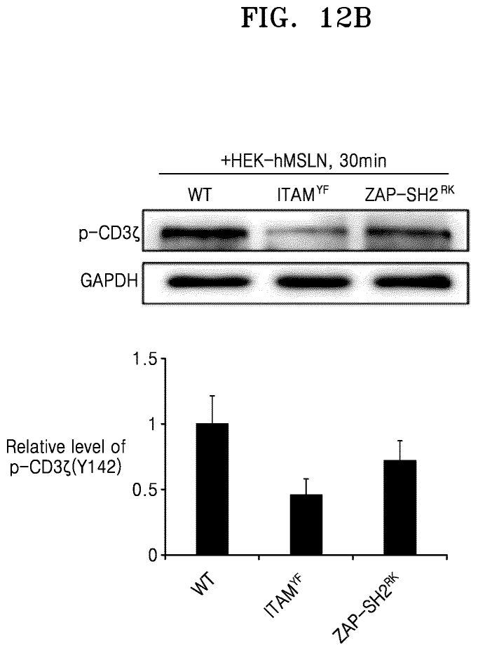

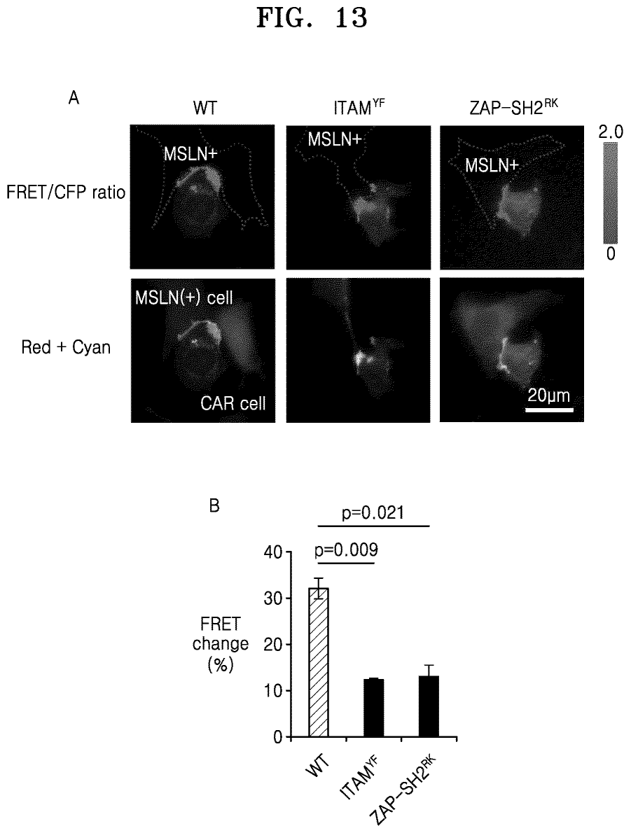

By using the FRET-based CAR activity sensor with the optimized linkers, the cancer antigen binding and changes in the CAR activity in a live cell state were measured based on FRET signals. A wild-type (WT) (SEQ ID NO: 11) sensor and ITAM^{YF} and ZAP-SH2^{RK} variant sensors wherein the first linker is ER/K and the second linker is GSG(7) were prepared, and changes in the FRET ratio in co-culture with cancer antigens were measured. The results are shown in FIGS. 12 and 13. FIGS. 12A and 12B show a result of western blotting detecting the FRET changes over time and the phosphorylation of the ITAM domain in co-culture of cells expressing the WT biosensor and cancer cells expressing mesothelin, and FIG. 13 shows the detection of FRET signals in co-culture of cells expressing the WT biosensor, the ITAM^{YF} or ZAP-SH2^{RK} variant sensors and cancer cells expressing mesothelin.

While the WT sensor showed an increase of about 30%, the ITAM^{YF} and ZAP-SH2^{RK} variant sensors were found to show a slight change in the FRET ratio, about 10%. Accordingly, it was found that the linker optimization increased the change in the FRET ratio without affecting the antigen binding of the CAR-FRET biosensor and consequent activation of T cells.

(3) Spacer

In the FRET-based CAR biosensor, the antigen-binding receptor domain on the surface of the cell is connected to the ITAM domain, the FRET domain, and the ZAP-SH2 domain in the cytoplasm, through the transmembrane domain, and the transmembrane domain and the antigen-binding receptor domain may be connected to each other through a spacer which serves as a hinge. Even for an antigen-binding receptor domain of a CAR with high binding affinity for a target antigen, actual binding can occur only when the antigen-binding receptor domain of the CAR is accessible to the target antigen. When cancer cells expressing the target antigen and T cells expressing CAR for the antigen are in contact, a contact site such as an immune synapse, is formed and the binding of the target antigen to the CAR occurs. However, when the target antigen is hidden by other receptors or structures on the surface of the cancer cell or is present so close to the cell membrane that it is inaccessible to the CAR, the binding of the target antigen to the CAR cannot occur.

In order to enable the CAR having binding affinity for a target antigen to effectively bind to the target antigen in the actual cellular environment, the effect of the length of the spacer in the CAR-FRET biosensor on the CAR activity was analyzed. When the spacer of the CAR is too short, the CAR may not bind to a target cancer antigen, whereas, when the spacer of the CAR is too long, the immune synapse may not be effectively formed, resulting in failure to induce the CAR activity.

In order to select the optimized spacer length for MSLN(M5)-CAR, a CAR spacer library was prepared in which the CD8 hinge (Hinge 4: SEQ ID NO: 7), the existing spacer was modified in lengths. Among this library, the effects of the spacers, i.e., Hinges 1 to 5, on the CAR activity were analyzed.

TABLE 3

| Name | Sequence (SEQ ID NO) |
|---|---|
| Hinge 1 | GGAVHTRGLDFACD |
| | (SEQ ID NO: 22) |
| Hinge 2 | RPEACRPAA-GGAVHTRGLDFACD |
| | (SEQ ID NO: 21) |
| Hinge 3 | TIASQPLSL-RPEACRPAA- |
| | GGAVHTRGLDFACD |
| | (SEQ ID NO: 20) |
| Hinge 4 | TTTPAPRPPTPAP-TIASQPLSL- |
| | RPEACRPAA-GGAVHTRGLDFACD |
| | (SEQ ID NO: 7) |
| Hinge 5 | TIASQPLSLR-TTTPAPRPPTPAP- |
| | TIASQPLSL-RPEACRPAA- |
| | GGAVHTRGLDFACD |
| | (SEQ ID NO: 19) |

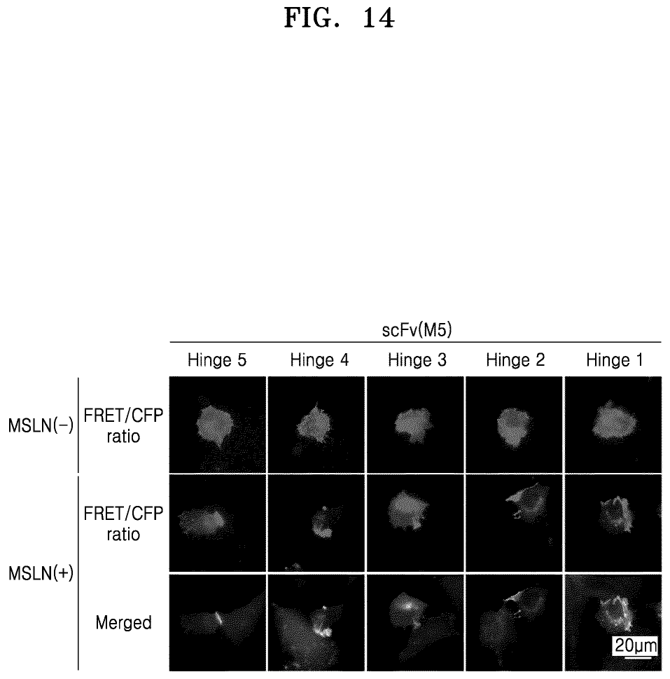

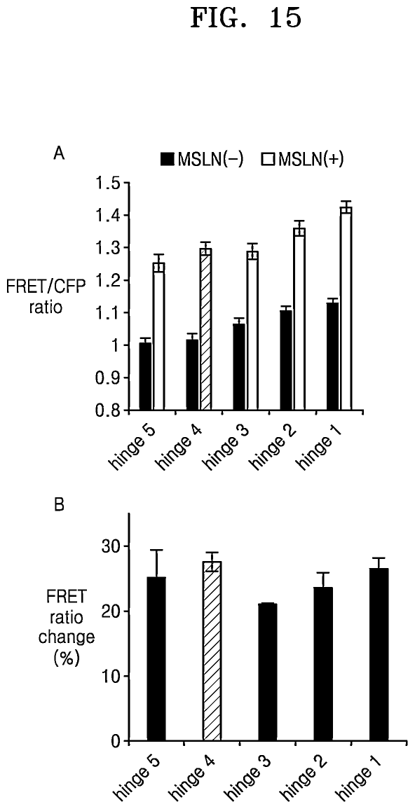

Cells expressing the WT CAR-FRET biosensor (Hinge 4) used in (2) and variants thereof (Hinge 1, Hinge 2, Hinge 3, and Hinge 5) in which hinge 4 is replaced with hinges 1 to 3 and 5, respectively, were co-cultured with either cells expressing mesothelin (MSLN(+)) or cells not expressing mesothelin (MSLN(−)), and then, a change in the FRET ratio was measured. The results are shown in FIGS. 14 and 15.

It was found that the FRET signals were affected by the length of the spacer. Hinge 1, a variant of MSLN(M5)-CAR which has a short spacer, was also able to access to a cancer antigen, and thus showed strong CAR activity induced by the cancer antigen-binding. However, in the case with the short CAR spacer (Hinge 1), the basal FRET level before antigen binding, that is, the tonic signal, was also high. A biosensor having high basal FRET level before antigen binding may show low sensitivity to antigen stimulation. Therefore, a biosensor with a low tonic signal, which is the FRET level before antigen binding, and a large change in the FRET ratio upon antigen binding may be an optimal biosensor for screening the CAR activity. Through the measurement of the FRET ratio by the spacer variant library, it was found that, when the spacer was Hinge 4, the signal before antigen binding was low and the change in the FRET ratio upon the antigen binding was the largest, and thus Hinge 4 was selected.

In this Example, by using the CAR-FRET biosensor variants, it was found that an optimized CAR vector that 1) binds to a cancer antigen and 2) induces maximum activation of CAR-T cells can be selected by comparing FRET changes of the biosensors having spacers of different lengths and scFvs to different epitopes of a cancer antigen. Unlike conventional methods, the CAR activity screening using the CAR-FRET biosensor according to the present disclosure considers not only the scFv domain but also the effective spacer length, so that the optimal domain combination of the CAR vector that responds most effectively to cancer cells can be efficiently selected in a live cell environment.

### Example 3. Detection of CAR Activity in Live Cells

This Example was to check whether the same results could be obtained in the actual Jurkat cells by using the sensor version finally designed and selected through optimization in Examples 1 and 2.

The work mechanism was confirmed by expressing the CAR vector in Jurkat cells, instead of HEK293 cells. Specifically, by using Jurkat cells expressing the WT CAR-FRET sensor and the CAR-FRET variant sensors, ITAM^{YF} and ZAP-SH2^{RK} variants in which residues involved in the phosphorylation of the ITAM domain and consequent binding of the ZAP-SH2 domain thereto were substituted, the work of the CAR-FRET biosensor and its mechanism were confirmed, wherein the WT CAR-FRET sensor includes scFv for M5 of mesothelin.

(1) Co-Culture Imaging

After the Jurkat cells were counted and prepared at 1×10⁶ cells/ml, the cells were washed once with PBS. For transfection, 10 μg of the CAR-FRET sensor expression vectors, each including the WT, the ITAM^{YF} or ZAP-SH2^{RK} variant sensor was added to an antibiotic-free RPMI1640 medium, and electrical stimulation was applied to the Jurkat cells by using the Neon electroporation system (1,500 V, 10 ms, 3 pulses). Then, the resulting Jurkat cells were transfected by culturing overnight at a temperature of 37° C. and 5% CO₂.

On the next day, the cells were spread on a poly-L-lysine-coated glass-bottom dish at a density of 1×10⁶ cells/mi and incubated for adhesion for 2 hours. To observe FRET changes in the co-culture, cell imaging was performed at 37° C. and 5% CO₂ by using a Nikon Ti-E inverted microscope. K562-hMSLN cells were inoculated into the Jurkat cell culture dish expressing the biosensor, and FRET images were obtained therefrom after 30 minutes.

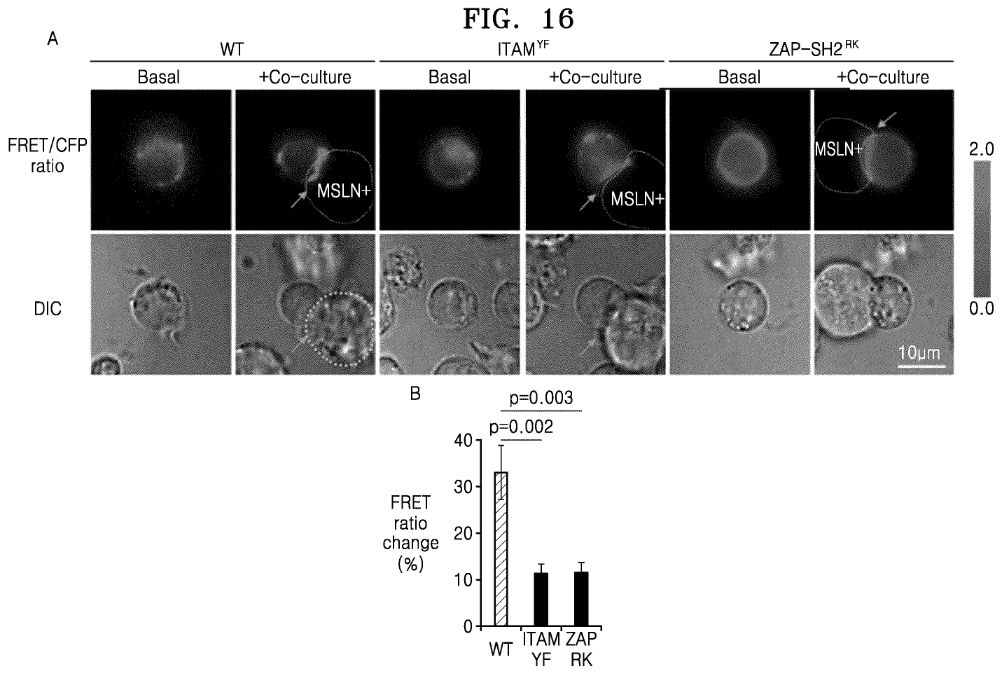

After removing the background signal by using the NIS program (Nikon), the fluorescence intensities of the ECFP and FRET channels at the synapse where the biosensor-expressing cells and the hMSLN-expressing cells were bound, and the FRET/ECFP ratio was obtained therefrom. The FRET/ECFP ratio for each group was calculated and quantified, and the difference in the FRET/ECFP ratio was graphed. The results are shown in FIG. 16. In FIG. 16, A shows fluorescence imaging anc B shows FRET ratio change for WT, ITAM^{YF}, and ZAP-SH2^{RK}.

As in the results in the HEK293 cells, it was found that the FRET ratio increased by about 30% at the synapse during the co-culture with cancer antigen-expressing cells, while the ITAM^{YF} and ZAP-SH2^{RK} variant sensors showed a low increase of about 10%. The increase of about 10% is interpreted due to dense gathering of the CAR-T cells and the cancer antigens in a narrow synaptic space.

Therefore, in predicting the CAR activity by FRET analysis, a CAR can be determined to be active when the FRET change is over 10%, which is shown by a negative variant, for example, 10% to 30%. A negative variant refers to the variant showing no or insufficient CAR activity.

(2) Western Blot

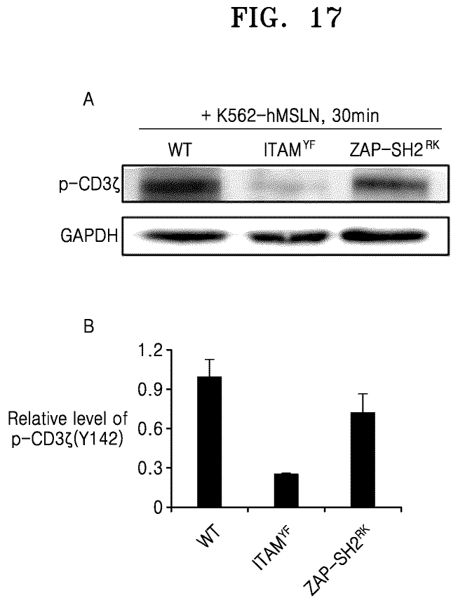

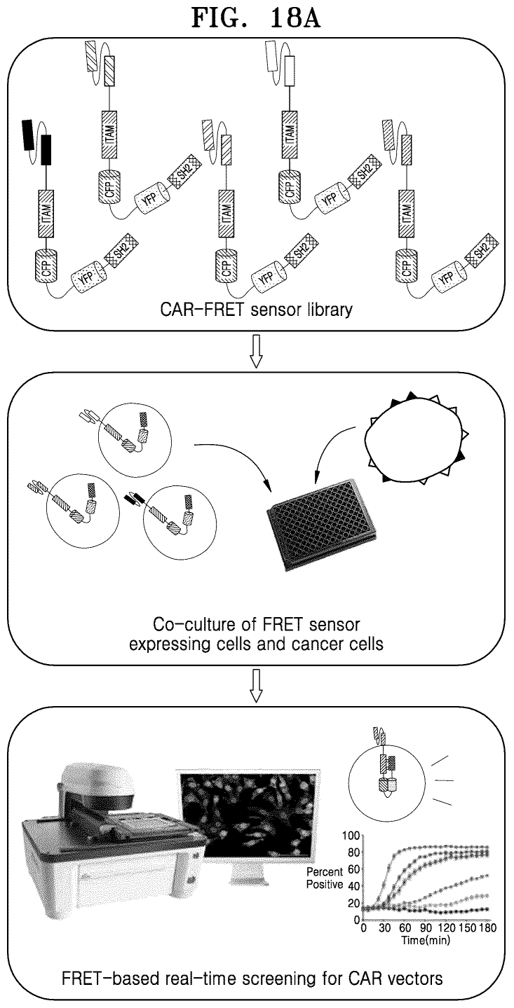

As described in Section (1), the Jurkat cells transfected to express the CAR-FRET biosensor or the biosensor variant were co-cultured with K562-hMSLN cells for 30 minutes, and then proteins were extracted and the amount of the proteins was quantified by BCA analysis. For western blotting, SDS-PAGE was performed by using 20 μg of each protein, and blots were added to a primary antibody solution containing anti-phospho CD3 (Cell signaling, #67747) and anti-GAPDH (Santacruz, #5c47724) and cultured overnight at a stirrer at 4° C. On the next day, the resulting blots were washed with TBS-T three times for 15 minutes each, and blots were added to a secondary antibody solution and incubated at a stirrer for 2 hours at room temperature. The resulting blots were washed with TBS-T buffer three times for 15 minutes each and then treated with an ECL solution to detec signals. The results are shown in FIG. 17.

### Example 4. Construction of Live FRET Cell Imaging-Based CAR Activity High Throughput Screening (HTS)

(1) Construction of Live FRET Cell Imaging-Based CAR Activity HTS System

In this Example, an HTS system capable of screening CAR-FRET biosensors for CAR activity in live cells with high efficiency through imaging was constructed.

The HTS system was configured to detect and read FRET signals upon binding of CAR to a cancer antigen via imaging by co-culturing cells expressing the CAR-FRET sensor library with cancer cells.

First, MSLN-2A-mCherry was prepared to determine the expression level of mesothelin (MSLN) by mCherry fluorescence, and quantitative relationship between the expression of mesothelin and the mCherry fluorescence intensity was established.

In addition, by co-culturing cells expressing cancer antigens at various levels with cells expressing the FRET-based CAR sensor, the FRET levels were quantified by the level of cancer antigen.

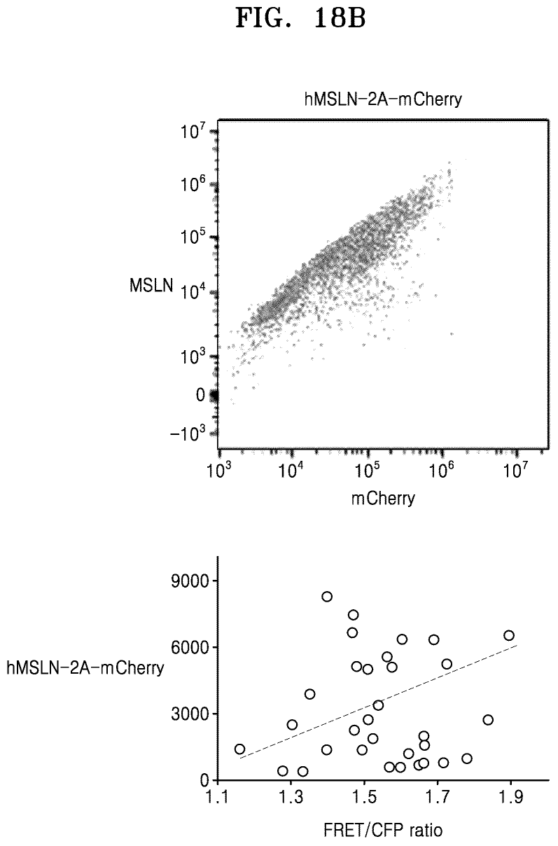

The real-time signals in the co-culture can be detected by measuring the expression of cancer antigens and consequent FRET ratios, and thus the CAR-FRET sensor library can be screened during co-culture with cancer antigen-expressing cells. FIG. 18 schematically shows an HTS-based screening method according to an embodiment of the present disclosure. FIG. 18A shows the configuration of the screening method based on the live FRET cell imaging-based CAR activity HTS system, and FIG. 18B shows quantitative relationship between expression of cancer antigen and fluorescence that are the basis of the HTS system and quantification of FRET ratios in co-culture with cancer antigen-expressing cells.

(2) Intermolecular CAR-FRET Sensor

Preparation of Vector for Expressing Intermolecular CAR-FRET Sensor

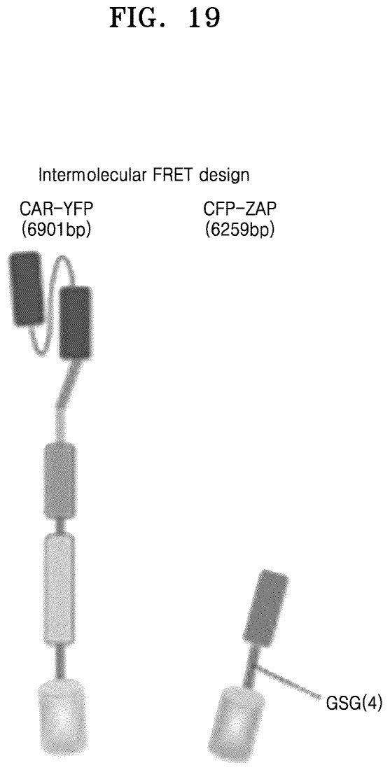

An intermolecular CAR-FRET sensor consisting of more than one molecule, each molecule is smaller than an intramolecular CAR-FRET sensor consisting of one molecule was designed. FIG. 19 shows the structure of the intermolecular CAR-FRET sensor.

The intermolecular CAR-FRET sensor was prepared by removing from the WT CAR-FRET sensor the ER/K linker between the fluorescence proteins and dividing into a FRET-receptor module (CAR-YFP: SEQ ID NO: 23) and a FRET-cytoplasmic module (CFP-ZAP: SEQ ID NO: 24), which are to be expressed together.

Live Imaging in Co-Culture with Cancer Cells

As described above, viruses expressing CFP-ZAP and CAR-YFP+CFP-ZAP, respectively, were prepared and transduced into Jurkat cells. The transduced Jurkat cells and cancer cells were co-cultured at a ratio of 1:1 in a 96-well dark-plate. Then, real-time imaging was captured by using lionheart-FX automation microscope (Biotek) equipment. Within 30 minutes after co-culture, fluorescence imaging was captured for each filter by using CFP, YFP, and FRET filters, and analyzed. After transduction with viruses each expressing the FRET-receptor module and FRET-cytoplasmic module, the Jurkat cells expressed the CAR-FRET biosensor, and changes in the FRET ratio could be detected in co-culture with cancer cells.

Linker Optimization

In order to optimize the length of linkers connecting the receptor module and the cytoplasmic module of the intermolecular CAR-FRET sensor, variants including one of the linkers from GSG(3) to GSG(8) were prepared, and the GSG (4) version was selected as the final version as the variant including the linker showed the highest change in FRET ratio compared to the basal level upon stimulation by Lck overexpression and the linker was the shortest.

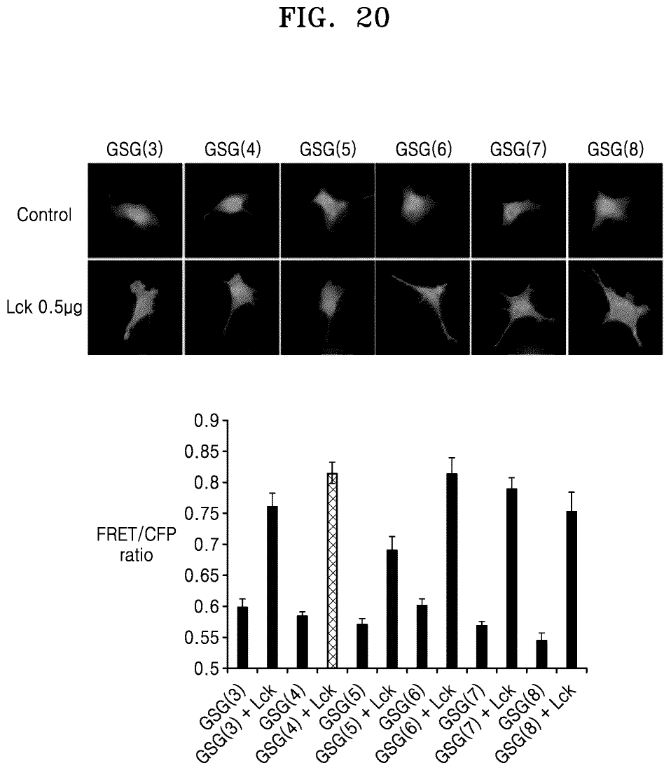

FIG. 20 shows the results of measuring the FRET signals by the linker variants.

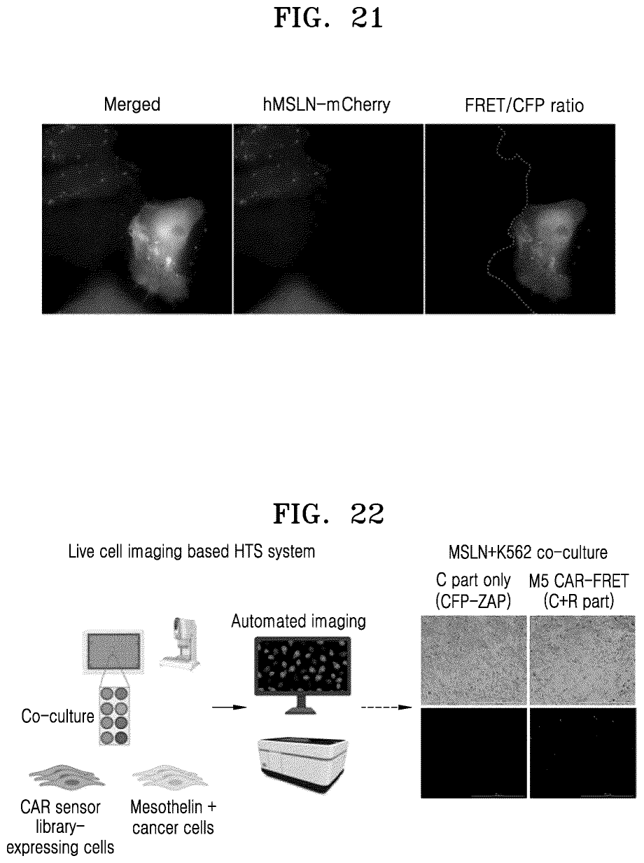

The GSG(4) version of the intermolecular CAR-FRET sensor as the final version was used to measure changes in the FRET ratio when co-cultured with cancer antigen expressing cells. It was found that the FRET ratio specifically increased at the site where cancer antigens bind to the CAR. The results are shown in FIG. 21.

HTS System

By using live cell imaging-based HTS equipment, imaging of co-culture of cells expressing the intermolecular CAR-FRET sensor and cells expressing mesothelin was performed. As a result, it was found that the FRET signal was higher in the group in which the CAR was formed by expressing the FRET-cytoplasmic module and the FRET-receptor module together, than that in the group in which only the FRET-cytoplasmic module (CFP-ZAP) was expressed. The results are shown in FIG. 22.

These results showed the possibility of constructing the imaging-based HTS system for screening massive CAR-FERT sensor library.

### Example 5. Screening and Preparation of Novel Mesothelin-Targeting CAR-T Cells with Improved Activity Compared to Existing CARs

In this Example, a mesothelin (MSLN)-targeting scFv antibody library of various affinities was prepared. Then, by using the CAR-FERT sensor according to an embodiment of the present disclosure, CARs with improved activity compared to the existing CARs were screened and CAR-T cells were prepared to confirm the CAR activity.

(1) Preparation of M5 scFv Variant Library

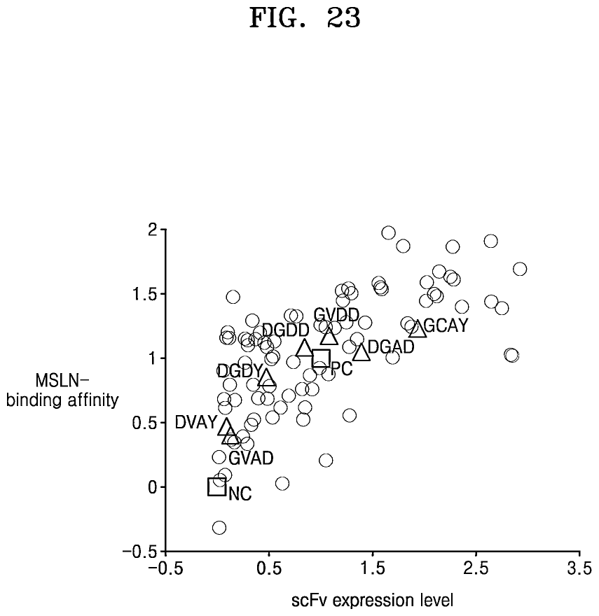

To prepare a library of M5 scFv variants with different affinities, a mutation was introduced by using a degenerated codon to the DFDY sequence of the heavy chain CDR3 (HCDR3) region of the M5 scFv (SEQ ID NO: 10) targeting mesothelin. The degenerated codons were introduced into the template antibody gene according to the Gibson assembly method, and each plasm id DNA library was transformed into E. coli BL21 (DE3) for protein expression. Then, 92 single clones were randomly selected from the antibody library and cultured in a 96 deep well plate. After culturing at 37° C. for about 16 hours, the cells were subcultured at 1/200 dilution for about 2 hours, and then, with addition of 0.5 mM IPTG to induce the expression of proteins, the cells were cultured at 25° C. for about 16 hours. The culture fluid containing the variant was added to each well of a 96-well plate and cultured at 4° C. for 16 hours. After blocking, the expression levels of antibodies were measured by using anti-His antibody-conjugated HRP, and the antigen binding affinity was measured by using biotinylated mesothelin and streptavidin-HRP. The randomly selected antibody clones each were found to showed different expression levels and different antigen-binding affinity, and seven M5 scFv variants (i.e., DVAY, GVAD, DGDY, DGDD, GVDD, DGAD, and GCAY) with various affinities ranging from high to low affinity were selected. The results are shown in FIG. 23.

(2) Individual Clonal Analysis of M5 scFv Variant Library

In order to measure the affinity of the WT M5 scFv (DFDY) and the selected seven scFv variants (i.e., DVAY, GVAD, DGDY, DGDD, GVDD, DGAD, and GCAY) of Section (1), for each clone, the cells were cultured at 37° C. until the OD600 of the culture fluid reached 0.6 to 0.8. Then, 0.5 nM IPTG was added to the culture to induce the expression of protein, and the cells were further cultured at 25° C. for 16 hours. Afterwards, the affinity of the purified M5 scFv variants was measured by ELISA.

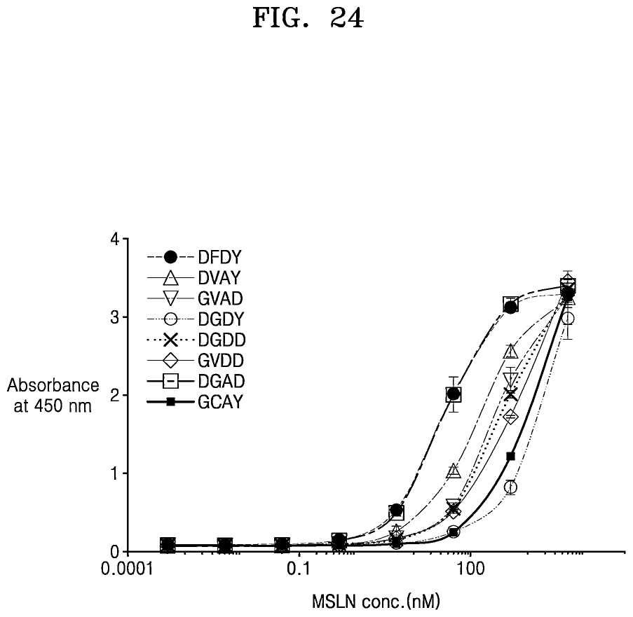

Specifically, each well of the 96-well immune plate was coated with 100 ng of the scFv variants produced as recombinant proteins at 4° C. for 16 hours. After adding 100 μl of pH 7.4 PBS supplemented with 3% bovine serum albumin (BSA) to each well, the cells were incubated at room temperature for 1 hour, and the plate was washed three times with 350 μl of PBS-T. The biotinylated hMSLN recombinant protein was diluted to 1 μM, 200 nM, 40 nM, 8 nM, 1.6 nM, and 0.32 nM in the pH 7.4 PBS, and reacted at room temperature for 1 hour with each of the coated scFv antibodies. Afterwards, the cells were washed three times with 350 μl of PBS-T per well. Then, 50 μl of HRP-conjugated streptavidin diluted at 1:15,000 in the pH 7.4 PBS was added to the cells followed by incubation of the cells at room temperature for 1 hour, and washing three times with 350 μl of PBS-T. After adding 50 μl of TMB, the cells were incubated at room temperature for 15 minutes, the reaction was terminated by adding 50 μl of 2 M H₂SO₄, and the absorbance at 450 nm was measured. The results are shown in FIG. 24.

(3) Preparation of M5 scFv Variant CAR-FRET Sensor Library

To prepare a CAR-FRET sensor library of seven M5 scFv variants with different affinities, cloning was performed by replacing the scFv region (SEQ ID NO: 10) of the prototype CAR-FRET sensor (SEQ ID NO: 11) with scFv variants. Here, the remaining fragment obtained by removing the CAR region by using a restriction enzyme in the CAR-FRET vector was purified and used as a vector. Each scFv variant and the CAR region except for the scFv portion were amplified by PCR, purified, and then used as separate inserts. The prepared vectors and inserts were ligated by using In-Fusion technology, and then transformed into E-coli DH5α. A colony was selected and placed in a culture medium containing antibiotics and cultured in a shaker at 37° C. for about 16 hours. Then, plasm id DNA was extracted therefrom by using a mini-prep kit. Plasm id DNA was confirmed by DNA sequencing.

(4) Co-Culture Imaging

(4-1) HEK293 Cell

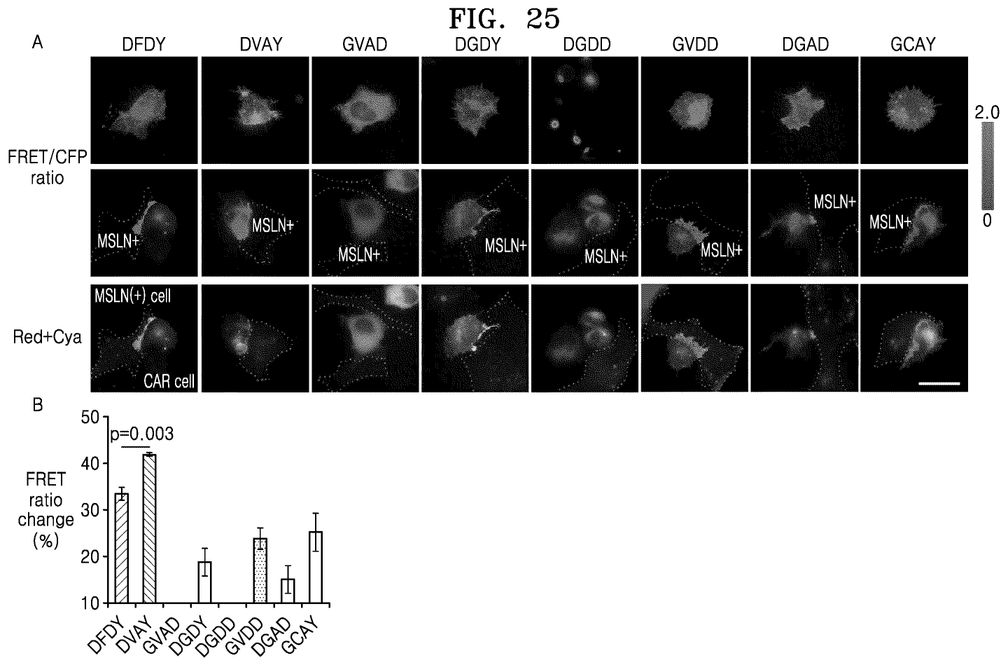

As described in Section (2-1) of Example 1, the M5 scFv variant CAR-FRET sensor library obtained in Section (3) was transfected into HEK293 cells and co-cultured with HEK293A cells transfected with the hMSLN-mCherry expression vector. (A) in FIG. 25 shows real-time FRET imaging.

In addition, after removing the background signal by using the NIS program (Nikon), the fluorescence intensities of the ECFP and FRET channels were measured at the synapse where the sensor-expressing cells and the hMSLN-expressing cells were bound, and the FRET/ECFP ratio was obtained therefrom. The FRET/ECFP ratio for each group was calculated and quantified, and the difference in the FRET/ECFP ratio was graphed. (B) in FIG. 25 shows the changes in the FRET ratio.

In the imaging, the GVAD variant and the DGDD variant were found to have poor expression, and thus were excluded from the analysis. The remaining variants were found to be different from each other in terms of the expression level and basal FRET ratio value. The variants (GAVD, DGDD) with poor expression levels and patterns were found failed to bind to the antigen even during co-culture. One variant (DVAY) with a higher change in the FRET ratio than the WT DFDY and one variant (GVDD) with an insignificant change in the FRET ratio were selected and used thereafter for a comparative experiment.

(4-2) Jurkat Cell

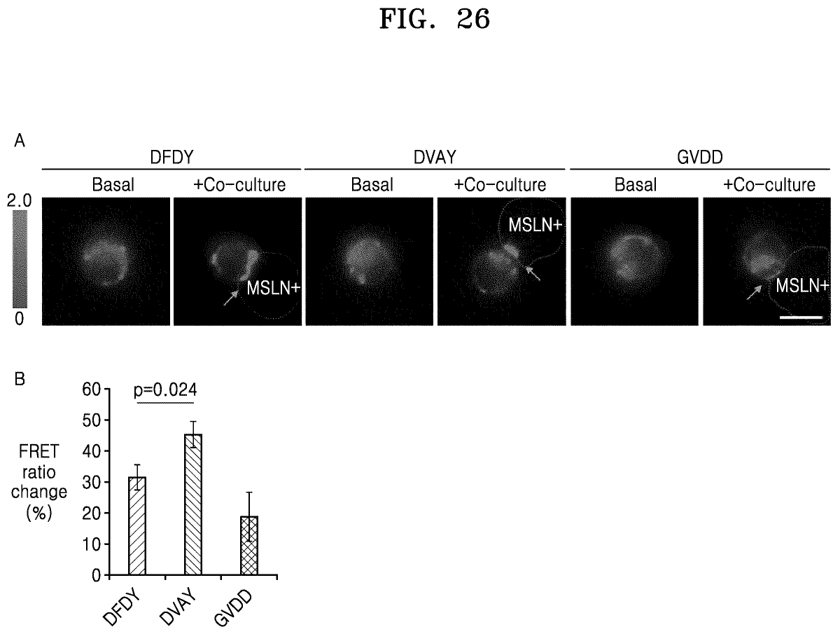

As described in Example 3, the CAR-FRET sensor expression vectors containing DFDY, DVAY, and GVDD scFv variants were transfected into Jurkat cells by electroporation, and K562-hMSLN cells were plated in a culture dish of the Jurkat cells expressing the sensor. 30 minutes later, FRET images were obtained. The results are shown in FIG. 26(A).

After removing the background signal by using the NIS program (Nikon), the fluorescence intensities of the ECFP and FRET channels at the synapse where the biosensor-expressing cells and the hMSLN-expressing cells were bound, and the FRET/ECFP ratio was obtained therefrom. The FRET/ECFP ratio for each group was calculated and quantified, and the difference in the FRET/ECFP ratio was plotted ((B) of FIG. 26).

As in the results in the HEK293 cells, WT DFDY was found to have about 30% increase in the FRET ratio at the synapse in co-culture with cancer antigen expressing cells, DVAY showed an increase by about 45%, and GVDD showed an increase by about 18%.

(5) CD69

As described in Section (1) of Example 2, in order to confirm whether the detection of FRET signals by the CAR-FRET biosensor reflected the activation of T cells subsequent to the cancer antigen-binding, the correlation with signals of CD69, a marker of the T cell activation, which is important for signal transduction, was analyzed. For this, a CAR variant was prepared by removing from the WT CAR FRET sensor the fluorescence protein and linker for the measurement of FRET signals. CAR-Jurkat cells including DFDY, DVAY, or GVDD scFvs were co-cultured with cells overexpressing cancer antigens, and then, CD69 signals measured therefrom were compared with FRET signals.

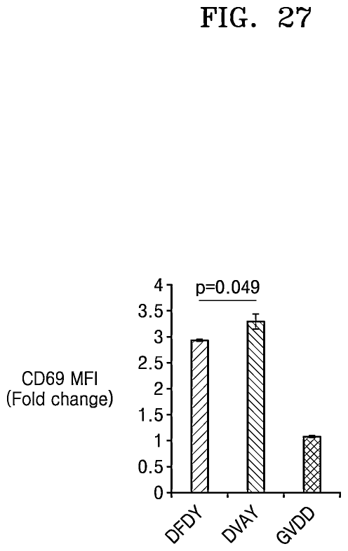

The detection of FRET signals by the CAR-FRET biosensor was found to be quite similar to the expression of CD69, a marker of the T cell activation. The results are shown in FIG. 27. It was found that the expression level of CD69 was higher in DVAY than in WT DFDY. Therefore, it was verified that the live FRET-based CAR biosensor can be utilized to screen various CAR variants for CAR activity.

(6) Affinity measurement (SPR) of DFDY, DVAY, and GVDD

To measure the affinity of the selected variants, surface plasmon resonance (SPR) was performed by using a BIAcore T200 device. For analysis, hMSLN was immobilized on a CM5 sensor chip by using an amine coupling method. Experiments were performed by using HBS-EP buffer, and diluted samples of each scFv variant were injected at a rate of 30 μl/min for 120 seconds and dissociated for 5 minutes. After the first experiment, 5 mM NaOH and 0.5 M arginine at pH 8.0 were sequentially injected for 30 seconds to regenerate the chip. For each scFv variant, three separate experiments were averaged to determine a dissociation constant, and each dissociation constant was determined by using a 1:1 Langmuir model in BIAevaluation 3.2 software.

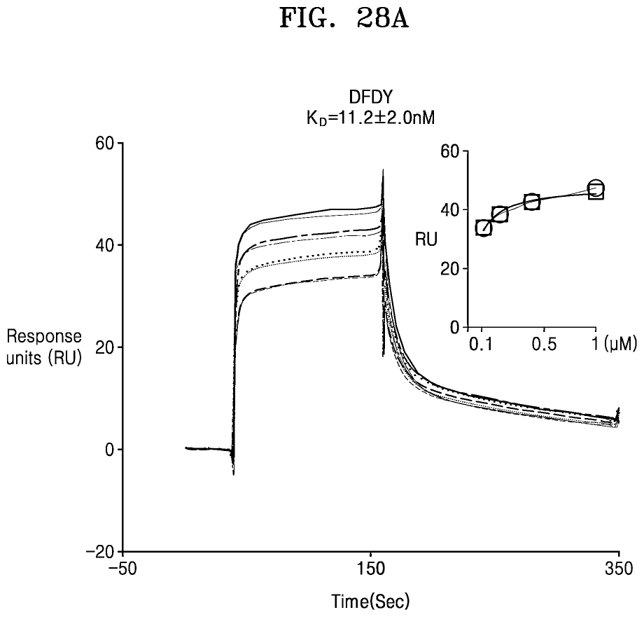

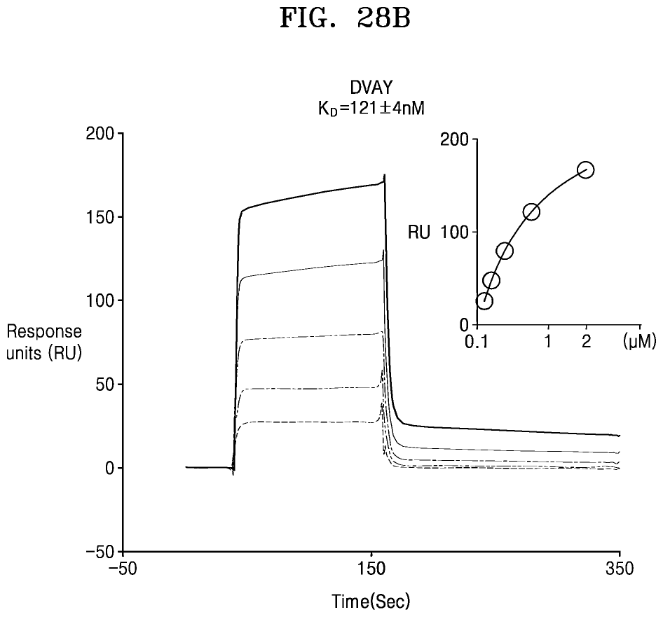

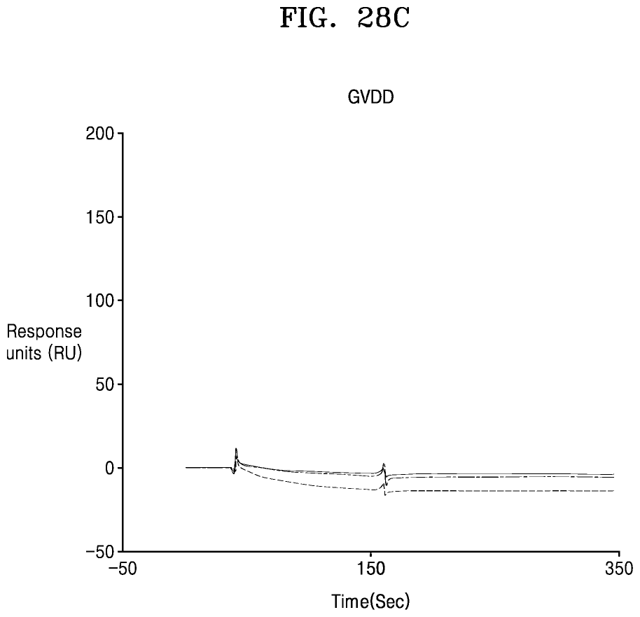

All three variants showed similar results to those of ELISA, with DFDY affinity measured to be 11.2±2 nM, DVAY affinity to be 121±4 nM, and GVDD affinity to be very low. The results are shown in FIG. 28.

(7) Preparation of WT(DFDY) and DVAY CAR Vectors and CAR-T Cells

For production of lentivirus to be used in the preparation of CAR-T cells, 25 μg of lentiviral vector and a packaging plasmid mixture (CAR-T transfer vector: MD2.G: pMDLg/pRRE: pRSV-Rev at a ratio 2:1:1:1) was transfected into Lenti-X 293 T cells by using lipofectamin3000 (Thermo Fisher Scientific), when Lenti-X 293T cells reached 90% confluency in a cell culture dish. On Day 1 and Day 2 after the transfection with the plasm id mixture, the cell culture fluid was recovered to secure the virus secreted into the cell culture. After centrifugation at 453×g for 3 minutes and purification with a 0.45 μm filter, the debris was removed and the resulting cell culture fluid was concentrated 100 times by centrifugation at 12,700×g at 4° C. for 120 minutes.

For the preparation of CAR-T cells, peripheral blood mononuclear cells (PBMCs) were isolated from heparinized peripheral blood by using Ficoll-Histopaque gradient (1.077 g/mL: GE Healthcare Life Sciences, Piscataway, NJ), and then frozen. After thawing the PBMCs, the cells were pre-cultured at 37° C. for 3 hours, and then used in the experiments. The cells were cultured in an AIM-V medium (Thermo Fisher Scientific) supplemented with antibody recognizing human CD3 (clone OKT3; Thermo Fisher Scientific, 10 μg/mL) and recombinant human IL-2 (200 IU/mL; Peprotech, Cranbury, NJ, USA), at 37° C. and 5% CO₂ conditions for 48 hours, thereby activating T cells. After the activation of T cells, the T cells were infected with lentivirus at a multiplicity of infection (MOI) of 7 and cultured for 48 hours. By culturing the lentivirus-infected cells for 10 to 11 days with addition of IL-2 (200 IU/mL) twice a week, the CAR-T was established for use in the evaluation of functions of the CAR-T cells.

(8) CAR-T Cell Function Evaluation

For in vitro efficacy analysis of DVAY variant CAR, which has lower affinity for recombinant mesothelin (MSLN) than WP (DFDY) but good FRET activity, a pLVX-hMSLN OX-cFLAG-IRES-Pgk in which MSLN is expressed under the control of the pgk promoter was prepared to prepare K562 cells with low expression of MSLN. Specifically, the pgk promoter was obtained from the pHR_Gal4UAS_IRES_mC_PGK_tBFP plasmid (Addgene, Watertown, MA, USA) to replace the EF-1 alpha promoter of the pLVX-hMSLN OX-cFLAG-IRES-Puro plasmid with the pgk promoter. The sequence accuracy of the recombinant plasmid was confirmed by using Sanger sequencing. Lentivirus was produced as described in Section (7), and K562 cells were infected with the lentivirus to prepare K562 cells with low MSLN expression.

K562 cells having different MSLN expression levels (low and high) were co-cultured with either the CAR-T cells including WT M5 scFv (DFDY) or the CAR-T cells including variant M5 scFv (DVAY) prepared in Section (7), and then, the expressions of CD137, CD25, and CD69, which are makers of CAR-T cell activation, the cytokine (IL-2, IFN-γ) productivity and cytotoxicity (apoptotic activity) to target cells were analyzed.

The CAR-T cells were co-cultured with target cells, MSLN+K562 cells or MSLN negative control, K562 cells (E:T ratio of 1:2) for 24 hours in the presence of an anti-human CD4 antibody (clone, RPA-T4) labeled with Brilliant Ultra Violet™ 805 (BUV805), and anti-human CD107a antibody (clone, H4A3) labeled with fluorescein isothiocyanate (FITC). After co-culturing the target cells with the CAR-T cells, to analyze the degree of the activation of CAR-T cells, the CAR-T cells were stained with antibodies, anti-human CD8 (clone, RPA-T8) labeled with BUV496, anti-human CD137 (clone, 4B4-1) labeled with BUV395, anti-human CD25 labeled with Brilliant Violet™ 711 (BV711), and anti-human CD69 (clone, FN50) labeled with PE (wherein the anti-human CD25 was purchased from BioLegend CD25, San Diego, CA, USA, and the others were purchased from BD Biosciences, San Joe, CA, USA). As a result, it was found that the expressions of T cell activation markers were higher in the DVAY CAR-T cells than in WT.

After co-culturing the CAR-T cells with K562 cells having different MSLN expression levels (high and low) at a E:T ratio of 1:2 for 6 hours, the cytokine (IL-2, IFN-γ) productivity was compared. To evaluate the cytokine productivity of the CAR-T cells, the co-culture as described above was conducted for 6 hours with an anti-human CD4 antibody labeled with BUV805 added. During the last 4 hours of the co-culture, the culture was conducted with BD Golgistop™ (monensin, BD Biosciences) and BD Golgiplug™ (brefeldin A, BD Biosciences) added for intracellular cytokine staining. After the culture, the cells were stained with anti-human CD8 antibody labeled with BUV496 and treated with fixation and permeabilization reagents, and then stained with antibodies, anti-human interferon (IFN)-γ (clone, B27; BD Biosciences) labeled with PE-cychrom 7 (Cy7), anti-human tumor necrosis factor (TNF)-α (clone, Mab11; eBioscience) labeled with allophycocyanin (APC), and anti-human interleukin (IL)-2 (clone, MQ1-17H12, BD Biosciences) labeled with BV711. The stained cells were obtained with a BD LSRFortessa™ flow cytometer (BD Biosciences) and analyzed by using a FlowJo 10.8.1 software (Tree Star, Ashland, OR, USA). The result showed that the DVAY variant CAR-T cells exhibited higher cytokine productivity than WT, as in the expression of activation markers.

In addition, to evaluate the target cytotoxicity (apoptosis ability), the aforementioned two types of CAR-T cells were prepared first. Then, K562 cells (2.5×10⁴) expressing MSLN were labeled with carboxy fluorescein diacetate succinimidyl ester (CFSE, 5 mM; Thermo Fisher Scientific) and co-cultured with the CAR-T cells at various ratios at 37° C. for 8 hours. After the culture, the cells were stained with 7-amino-actinomycin (7-AAD, BD Biosciences). The stained cells were obtained by using a BD LSRFortessa™ flow cytometer and analyzed by using a FlowJo 10.8.1 software. The ratio of apoptotic target cells positive for CFSE and 7-AAD was calculated after subtracting the spontaneous apoptosis ratio in the target cells. That is, the apoptotic rate of target cells was calculated as [(% experimental apoptosis−% spontaneous apoptosis)/(% maximal apoptosis−% spontaneous apoptosis)]×100. The spontaneous apoptosis refers to cell death by lysis of the target cells without the CAR-T cells, and the maximal apoptosis refers to cell death by treatment with 100 μl of 0.1% Tween-20 for maximal lysis of the cells.

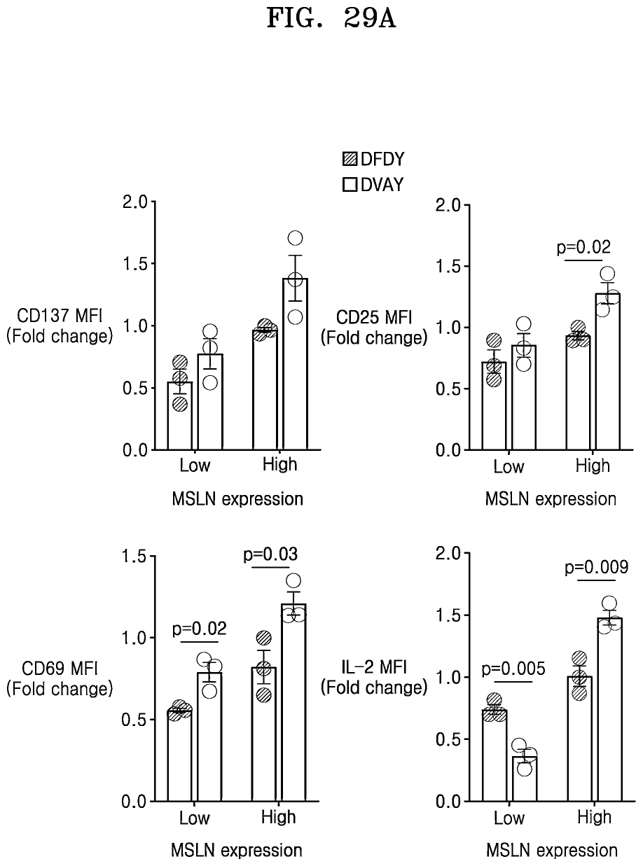

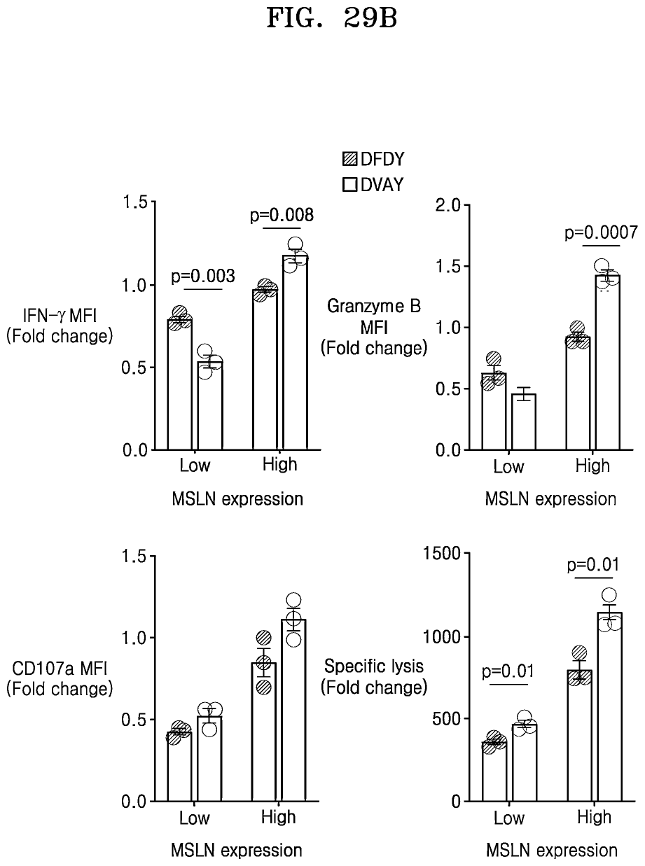

The cytotoxicity ability (apoptosis ability) of the CAR-T cells was evaluated by proportion of dead cells stained with 7-AAD, showing that the DVAY variant CAR-T cells had higher cytotoxicity than WT, as in the expression of activation markers and the cytokine productivity. FIGS. 29A and 29B show the results of measuring cytokine productivity and apoptotic activity of the wild-type M5 scFv (DFDY) and the variant M5 scFv (DVAY) screened according to an embodiment of the present disclosure.

These results show that scFvs with high affinity for antigens do not necessarily determine excellent activity of the CAR-T cells. That is, screening of the scFv of the CAR based solely on the binding affinity for antigens may actually lead to different results in the activity of the CAR-T cells, and thus, it is important to consider both the binding affinity to antigens and consequent intracellular signal transduction when screening CARs. In addition, it was verified that, as an HTS system to screen for various CAR variants, the CAR-FRET system capable of simultaneously measuring the antigen binding and intracellular signal transduction can be utilized.

A biosensor according to an embodiment of the present disclosure can bind to a target cancer antigen and subsequently induce activation of CAR-T cells so as to select an effective CAR vector showing a strong FRET value in a live cell environment, with high accuracy and efficiency. In this regard, the biosensor can be utilized for the development of effective CARs for CAR-T cell, a next-generation cancer immunotherapy.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A fluorescence resonance energy transfer (FRET)-based biosensor for sensing chimeric antigen receptor (CAR) activity, comprising: an antigen-binding receptor domain; a transmembrane domain; an immunoreceptor tyrosine-based activation motif (ITAM) domain; a FRET domain comprising a fluorescence donor and a fluorescence acceptor; and a ZAP-SH2 domain,
wherein binding of a target antigen to the antigen-binding receptor domain leads to phosphorylation of the ITAM domain, and the phosphorylated ITAM domain binds to the ZAP-SH2 domain so that a detectable FRET signal is generated in the FRET domain, thereby simultaneously detecting antigen binding and consequent activation of T cells to verify CAR activity.

2. The FRET-based biosensor of claim 1, wherein the biosensor further comprises a spacer between the antigen-binding receptor domain and the transmembrane domain and wherein the spacer is selected so that the antigen-binding receptor domain is accessible to a target epitope in a surface environment of cells expressing a target antigen.

3. The FRET-based biosensor of claim 1, wherein the ITAM domain is derived from CD3 zeta.

4. The FRET-based biosensor of claim 1, wherein the FRET domain comprises a fluorescence donor protein and a fluorescence acceptor protein, wherein the fluorescence donor protein and the fluorescence acceptor protein are connected via a first linker, and one end of the FRET domain and the ZAP-SH2 domain are connected via a second linker.

5. The FRET-based biosensor of claim 4, wherein the first linker is an ER/K linker of SEQ ID NO: 18, the second linker is a GSG(7) linker of SEQ ID NO: 6, the transmembrane domain has an amino acid sequence of SEQ ID NO: 8, the ITAM domain has an amino acid sequence of SEQ ID NO: 1, the FRET domain includes yellow fluorescent protein (YFP) and cyan fluorescent protein (CFP), and the ZAP-SH2 domain has an amino acid sequence of SEQ ID NO: 2.

6. The FRET-based biosensor of claim 1, wherein the antigen-binding receptor domain is a single-chain variable fragment (scFv) for a cancer antigen, and the FRET domain includes yellow fluorescent protein (YFP) and cyan fluorescence protein (CFP), wherein, when the scFv binds to a cancer antigen, the ITAM domain is phosphorylated and the ZAP-SH2 binds to the phosphorylated ITAM domain to generate a FRET signal in the FRET domain, thereby detecting cancer antigen binding and consequent activation of T cells.

7. The FRET-based biosensor of claim 1, the FRET signal is measured by a FRET ratio or a change thereof.

8. The FRET-based biosensor of claim 1, wherein the FRET-based biosensor is present on a membrane of a live cell and is capable of verifying CAR activity in an actual cell environment when co-cultured with a cell expressing a target antigen.

9. The FRET-based biosensor of claim 1, wherein the antigen-binding receptor domain is a single-chain variable fragment (scFv) for a cancer antigen, and the FRET-based biosensor provides a tonic signal of a CAR by detecting a level of activation of T cells by the scFv in the absence of a cancer antigen.

10. The FRET-based biosensor of claim 1, wherein the FRET-based biosensor is in the form of a single fusion protein, or consists of a FRET-receptor module comprising an antigen-binding receptor domain, an ITAM domain, and a fluorescence donor or fluorescence acceptor of a FRET domain; and a FRET-cytoplasmic module comprising a ZAP-SH2 domain and a fluorescence donor or fluorescence acceptor not included in the FRET receptor module.

11. A nucleic acid encoding the FRET-based biosensor of claim 1.

12. The nucleic acid of claim 11, wherein the FRET-based biosensor consists of a FRET-receptor module and a FRET-cytoplasmic module, and the nucleic acid comprises: a first nucleic acid molecule encoding the FRET-receptor module; and a second nucleic acid molecule encoding the FRET-cytoplasmic module, wherein the FRET-receptor module comprises an antigen-binding receptor domain, an ITAM domain, and a fluorescence donor or fluorescence acceptor of a FRET domain; and the FRET-cytoplasmic module comprises a ZAP-SH2 domain and a fluorescence donor or fluorescence acceptor not included in the FRET receptor module.

13. A cell expressing the FRET-based biosensor of claim 1.

14. The cell of claim 13, wherein the cell comprises a vector encoding the FRET-based biosensor.

15. The cell of claim 13, wherein the cell comprises: a first vector comprising a first nucleic acid molecule encoding a FRET-receptor module; and a second vector comprising a second nucleic acid molecule encoding a FRET-cytoplasmic module, wherein the FRET-receptor module comprises an antigen-binding receptor domain, an ITAM domain, and a fluorescence donor or fluorescence acceptor of a FRET domain, and the FRET-cytoplasmic module comprises a ZAP-SH2 domain and a fluorescence donor or fluorescence acceptor not included in the FRET receptor module.

16. A method of screening for a chimeric antigen receptor (CAR) in a live cell by using the FRET-based biosensor of claim 1, the method comprising:
expressing a nucleic acid encoding the FRET-based biosensor in a cell;
contacting the FRET-based biosensor with a target antigen; and
measuring a change in FRET signals.

17. The method of claim 16, wherein the contacting of the FRET-based biosensor with the target antigen is performed by co-culturing cells expressing the FRET-based biosensor with cells expressing the target antigen.

18. The method of claim 16, wherein the measuring of the change in FRET signals comprises selecting a CAR as an effective CAR when the change in FRET signals is higher than a change in a CAR verified to bind to an antigen and activate T cells.

19. The method of claim 16, wherein the measuring of the change in FRET signals comprises selecting a CAR as an effective CAR when a change in FRET signals is 10% or more and is higher than a change in FRET signals before the contacting with the target antigen.

20. The method of claim 16, wherein the method is high-throughput screening (HTS) based on live cell imaging.


SEQUENCE LISTING (24 sequences)

SEQ ID NO: 1 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 257 aa, synthetic construct
PDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSS

SEQ ID NO: 3 - protein, 103 aa, synthetic construct
EFGSGGSGENLYFQGGSGEEEEKKKQQEEEAERLRRIQEEMEKERKRREEDEQRRRKEEEERRMKLEMEAKRKQEEEERKKREDDEKRKKKGSGGSGGSGGAP

SEQ ID NO: 4 - protein, 238 aa, synthetic construct
VSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSKLSKDPNEKRDHMVLLEFVTAAGITLGMDELYK

SEQ ID NO: 5 - protein, 239 aa, synthetic construct
MVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 6 - protein, 21 aa, synthetic construct
GSGGSGGSGGSGGSGGSGGSG

SEQ ID NO: 7 - protein, 45 aa, synthetic construct
TTTPAPRPPTPAPTIASQPLSLRPEACRPAAGGAVHTRGLDFACD

SEQ ID NO: 8 - protein, 24 aa, synthetic construct
IYIWAPLAGTCGVLLLSLVITLYC

SEQ ID NO: 9 - protein, 42 aa, synthetic construct
KRGRKKLLYIFKQPFMRPVQTTQEEDGCSCRFPEEEEGGCEL

SEQ ID NO: 10 - protein, 241 aa, synthetic construct
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 11 - protein, 1362 aa, synthetic construct
MALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRSGSGGSGGSGGSGVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSKLSKDPNEKRDHMVLLEFVTAAGITLGMDELYKEFGSGGSGENLYFQGGSGEEEEKKKQQEEEAERLRRIQEEMEKERKRREEDEQRRRKEEEERRMKLEMEAKRKQEEEERKKREDDEKRKKKGSGGSGGSGGAPMVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGITLGMDELYKLINGSGGSGGSGGSGGSGGSGGSGASPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSS

SEQ ID NO: 12 - protein, 21 aa, synthetic construct
MALPVTALLLPLALLLHAARP

SEQ ID NO: 13 - protein, 14 aa, synthetic construct
RSGSGGSGGSGGSG

SEQ ID NO: 14 - protein, 257 aa, synthetic construct
MALPVTALLLPLALLLHAARPDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKGGGGSGGGGSGGGGSQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSS

SEQ ID NO: 15 - protein, 253 aa, synthetic construct
QQQLEESGGGLVKPEGSLTLTCKASGFDLGFYFYACWVRQAPGKGLEWIACIYTAGSGSTYYASWAKGRFTISKASSTTVTLQMTSLAAADTATYFCARSTANTRSTYYLNLWGPGTLVTVSSGGGGSGGGGSGGGGSGGGGSDVVMTQTPASVSEPVGGTVTIKCQASQRISSYLSWYQQKPGQRPKLLIFGASTLASGVPSRFKGSGSGTEYTLTISDLECADAATYYCQSYAYFDSNNWHAFGGGTEVVV

SEQ ID NO: 16 - protein, 252 aa, synthetic construct
QEQLEESGGDLVKPGASLTLTCTASGFSFSSSYWICWVRQAPGKGLEWIACIYAGDGGATYDASWVNGRFSISKTSSTTVTLQMTSLTAADTATYFCARKGAAPTTYYYFNLWGPGTLVTVSSGGGGSGGGGSGGGGSGGGGSAYDMTQTPASVSAAVGGTVTIKCQASQSISTALAWYQQKPGQPPKVLIYAASNLASGVSSRFKGSGSGTEFTLTISDLECADAATYYCQQAATIINVDNVFGGGTEVVV

SEQ ID NO: 17 - protein, 98 aa, synthetic construct
GSGGSGGSGGSGGSGGSGEAAAKEAAAKEAAAKEAAAKEAAAKEAAAKAEAAAKEAAAKEAAAKEAAAKEAAAKEAAAKAGSGGSGGSGGSGGSGGSG

SEQ ID NO: 18 - protein, 73 aa, synthetic construct
EEEEKKKQQEEEAERLRRIQEEMEKERKRREEDEERRRKEEEERRMKLEMEAKRKQEEEERKKREDDEKRKKK

SEQ ID NO: 19 - protein, 55 aa, synthetic construct
TIASQPLSLRTTTPAPRPPTPAPTIASQPLSLRPEACRPAAGGAVHTRGLDFACD

SEQ ID NO: 20 - protein, 30 aa, synthetic construct
TIASQPLSLRPEACRPAAGGAVHTRGLDFA

SEQ ID NO: 21 - protein, 23 aa, synthetic construct
RPEACRPAAGGAVHTRGLDFACD

SEQ ID NO: 22 - protein, 14 aa, synthetic construct
GGAVHTRGLDFACD

SEQ ID NO: 23 - protein, 737 aa, synthetic construct
MALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRSGSGGSGGSGGSGVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSKLSKDPNEKRDHMVLLEFVTAAGITLGMDELYK

SEQ ID NO: 24 - protein, 513 aa, synthetic construct
MVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGITLGMDELYKLINGSGGSGGSGGSGASPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSS